(19) United States
(12) Patent Application Publication
(10) Pub. No.: US 2014/0031562 A1
(43) Pub. Date: Jan. 30, 2014

(54) Method for Forming Allylic Alcohols

(71) Applicants: Scott E. Denmark, Champaign, IL (US); Selena Milicevic, Urbana, IL (US); Son T. Nguyen, Cambridge, MA (US)

(72) Inventors: Scott E. Denmark, Champaign, IL (US); Selena Milicevic, Urbana, IL (US); Son T. Nguyen, Cambridge, MA (US)

(73) Assignee: The Board of Trustees of the University of Illinoi, Urbana, IL (US)

(21) Appl. No.: 13/060,793

(22) PCT Filed: Aug. 28, 2009
(86) PCT No.: PCT/US09/55363

§ 371 Date: May 7, 2013

Related U.S. Application Data
(60) Provisional application No. 61/092,898, filed on Aug. 29, 2008.

(51) Int. Cl. C07C 29/14; C07D 207/48; C07D 307/42; C07D 333/16; C07C 41/28; C07C 213/08

(52) U.S. Cl. CPC C07C 29/14; C07C 41/28; C07C 213/08; C07D 307/42; C07D 333/16; C07D 207/48
USPC 548/542; 568/813; 568/654; 564/443; 568/811; 568/808; 549/497; 549/78

(57) ABSTRACT

A method of performing a chemical reaction includes reacting an allyl donor and a substrate in a reaction mixture, and forming a homoallylic alcohol in the reaction mixture. The substrate may be an aldehyde or a hemiacetal. The reaction mixture includes a ruthenium catalyst, carbon monoxide at a level of at least 1 equivalent relative to the substrate, and water at a level of at least 1 equivalent relative to the substrate, and an amine at a level of from 0 to 0.5 equivalent relative to the substrate. The reaction mixture may also include a halide, and the equivalents of the amine may be similar to those of the halide. The reacting includes maintaining the reaction mixture at a temperature of at least 40° C. The method may be catalytic in metal, environmentally benign, amenable to large-scale applications, and applicable to a wide range of substrates.

### REFERENCE TO RELATED APPLICATIONS

This application claims the benefit of U.S. Provisional Application No. 61/092,898 entitled “Allylation Methods” filed Aug. 29, 2008, which is incorporated by reference in its entirety.

### BACKGROUND

Homoallylic alcohols are useful raw materials and/or intermediates for products including pharmaceuticals, fragrances, agricultural chemicals, and polymers. The alcohol and alkene functional groups can be transformed into a variety of other useful functional and structural groups. As depicted in Scheme 1, below, a homoallylic alcohol (C) may be formed by reacting a carbonyl-containing substance (A) with an allyl donor substance (B).

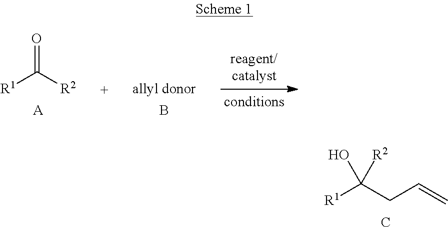

A variety of such carbonyl allylation reactions have been developed, with reports of yields greater than 80%, and of enantiomeric excess (ee) up to 98%. The benefits of conventional carbonyl allylation reactions include the diversity of reagent reactivity based on allylmetal reagents used in the reactions, and the degree of both diastereo- and enantioselectivity observed in some reactions.

Although many reagent variations are known, the reactions typically involve the use of discrete allylmetallic or non-metallic reagents, either directly or in combination with Lewis acidic or basic catalysts. In some cases, the allylmetallic reagent is prepared in situ by the combination of an allyl source such as a halide, an acetate or an alcohol, with a stoichiometric amount of a metal salt. A number of reports have described using one metal to catalyze the formation of the allylmetallic reagent, and a stoichiometric amount of a second metal or metallic reagent to turn over the catalyst. Only three methods are known to have been reported in which only catalytic amounts of metal reagents are used: the carbonyl-ene process, the hydrogenative coupling of dimethylallene and allyl acetate, and the ruthenium-catalyzed allylation of aldehydes using allyl acetate.

One example of a conventional carbonyl allylation method is the ruthenium catalyzed allylation of aldehydes. As depicted in Scheme 2, below, a homoallylic alcohol (F) is formed by reacting an aldehyde (D) as the carbonyl-containing substance with allyl acetate (E) as the allyl donor substance.

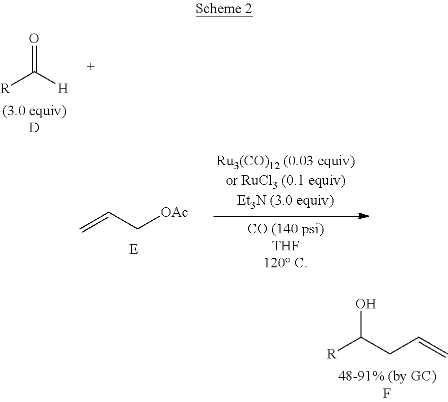

While this reaction is reported to provide good yields of aryl aldehydes, lower yields were provided with alkyl and alkenyl aldehydes. The reaction conditions for this method are relatively harsh, requiring a high pressure of carbon monoxide (CO) and a high reaction temperature, at which many aldehydes can degrade. The reaction uses a large excess of the potentially expensive aldehyde and requires a relatively large amount of the ruthenium catalyst, resulting in a significant amount of waste and an undesirably low atom efficiency. In addition, the large excess of triethylamine (Et₃N) can lead to undesirable side reactions, such as condensation of the aldehyde. However, mechanistic studies of the reaction have identified that the large amount of amine is necessary to provide hydrogen donation and to reduce the ruthenium.

The conventional methods for forming homoallylic alcohols have a number of disadvantages. Disadvantages of the conventional methods include the need for stoichiometric or excess amount of metallic or semimetallic reagents, which can cause problems in reaction workup and product purification, and which is a non-economic approach; the need for excess amounts of other, nonmetallic reagents; the need for expensive catalysts; the need for corrosive reagents and/or harsh reaction conditions; and/or applicability only to a limited range of carbonyl-containing substrates.

It would be desirable to form homoallylic alcohol products using a method that is more efficient, simpler to purify, more economical, and/or has less environmental impact than conventional carbonyl allylation methods. It would also be desirable to form a wider variety of homoallylic alcohol products by using a wider variety of carbonyl-containing substances and/or allyl donor substances than can be used in conventional carbonyl allylation methods.

### SUMMARY

In one aspect, the invention provides a method of performing a chemical reaction that includes reacting an allyl donor and a substrate in a reaction mixture, and forming a homoallylic alcohol in the reaction mixture. The substrate is an aldehyde or a hemiacetal. The reaction mixture includes a ruthenium catalyst, a halide, carbon monoxide at a level of at least 1 equivalent relative to the substrate, water at a level of at least 1 equivalent relative to the substrate, and an amine at a level of from 0.01 to 0.5 equivalent relative to the substrate. The reacting includes maintaining the reaction mixture at a temperature of at least 40° C.

Allylation methods including exposing a substrate to allyl reagent in the presence of a Ru-catalyst, wherein the Ru-catalyst is provided at 0.03 or fewer equivalents to the substrate are provided. Allylation methods including exposing a substrate to allyl reagent in the presence of a Ru-catalyst, the substrate, allyl reagent, and Ru-catalyst being comprised by a reaction mixture that is maintained at a temperature of less than 100° C. during the exposing are provided. Allylation methods including exposing a substrate to an allyl reagent in the presence of a Ru—X-catalyst (X=halide) and an amine, wherein the amine is provided at less than 1.0 equivalents to the substrate are provided. Allylation methods including exposing a substrate to an allyl reagent in the presence of a halide-free Ru-catalyst and one or both of a soluble halide or carboxylate salt are provided. Allylation methods including: forming a reaction mixture comprising a substrate, an allyl reagent, a Ru-catalyst, water, and CO; and maintaining a temperature of the reaction mixture above 40° C. are provided. Allylation methods including exposing a substrate to allyl reagent in the presence of a Ru-catalyst and CO, wherein the CO is provided at 1.0 or more equivalents to the substrate are provided.

The following definitions are included to provide a clear and consistent understanding of the specification and claims.

The term “homoallylic alcohol” means a substance having structural formula (I):

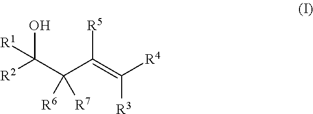

where R¹ is an organic group, and R²-R⁷ independently are H or an organic group. Preferably R² is H. More preferably R₂-R⁴ are H.

The term “group” means a linked collection of atoms or a single atom within a molecular entity, where a molecular entity is any constitutionally or isotopically distinct atom, molecule, ion, ion pair, radical, radical ion, complex, conformer etc., identifiable as a separately distinguishable entity. The description of a group as being “formed by” a particular chemical transformation does not imply that this chemical transformation is involved in making the molecular entity that includes the group.

The term “organic group” means a group containing at least one carbon atom.

The term “alkyl group” means a group formed by removing a hydrogen from a carbon of an alkane, where an alkane is an acyclic or cyclic compound consisting entirely of hydrogen atoms and saturated carbon atoms. An alkyl group may include one or more substituent groups.

The term “heteroalkyl group” means a group formed by removing a hydrogen from a carbon of a heteroalkane, where a heteroalkane is an acyclic or cyclic compound consisting entirely of hydrogen atoms, saturated carbon atoms, and one or more heteroatoms. A heteroalkyl group may include one or more substituent groups.

The term “alkenyl group” means a group formed by removing a hydrogen from a carbon of an alkene, where an alkene is an acyclic or cyclic compound consisting entirely of hydrogen atoms and carbon atoms, and including at least one carbon-carbon double bond. An alkenyl group may include one or more substituent groups.

The term “heteroalkenyl group” means a group formed by removing a hydrogen from a carbon of a heteroalkene, where a heteroalkene is an acyclic or cyclic compound consisting entirely of hydrogen atoms, carbon atoms and one or more heteroatoms, and including at least one carbon-carbon double bond. A heteroalkenyl group may include one or more substituent groups.

The term “aryl group” means a group formed by removing a hydrogen from a ring carbon atom of an aromatic hydrocarbon. An aryl group may by monocyclic or polycyclic and may include one or more substituent groups.

The term “heterocyclic group” means a group formed by removing a hydrogen from a carbon of a heterocycle, where a heterocycle is a cyclic compound consisting entirely of hydrogen atoms, saturated carbon atoms, and one or more heteroatoms. A heterocyclic group may include one or more substituent groups. Heterocyclic groups include cyclic heteroalkyl groups, cyclic heteroalkenyl groups, cyclic heteroalkynyl groups and heteroaryl groups.

The term “substituent group” means a group that replaces one or more hydrogen atoms in a molecular entity.

The term “halide group” means —F, —Cl, —Br or —I.

The term “allyl donor” means an alkene that can react with a carbonyl-containing substance (A in Scheme 1) to form a homoallylic alcohol. An allyl donor may have structural formula (II):

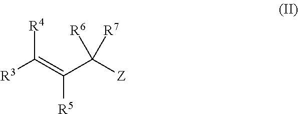

where R³-R⁷ may be H or an organic group, and where —Z is an alcohol group (—OH), a halide group, or an organic group.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1 represents a method of performing a chemical reaction.

FIG. 2 represents chemical structures, reaction schemes and product yields (in parentheses) for examples of reactions of various aldehyde substrates with allyl acetate to form homoallylic alcohols.

FIG. 3 represents chemical structures, reaction schemes and product yields (in parentheses) for examples of reactions of various hemiacetal substrates with allyl acetate to form homoallylic alcohols.

FIG. 4 represents a possible reaction pathway for the reaction of an aldehyde substrate with allyl acetate.

### DETAILED DESCRIPTION

A method of performing a chemical reaction includes reacting an allyl donor and an aldehyde or cyclic hemiacetal substrate in a reaction mixture, and forming a homoallylic alcohol in the reaction mixture. The method may provide one or more advantages, including being catalytic in metal, environmentally benign, amenable to large-scale applications, and applicable to a wide range of substrates.

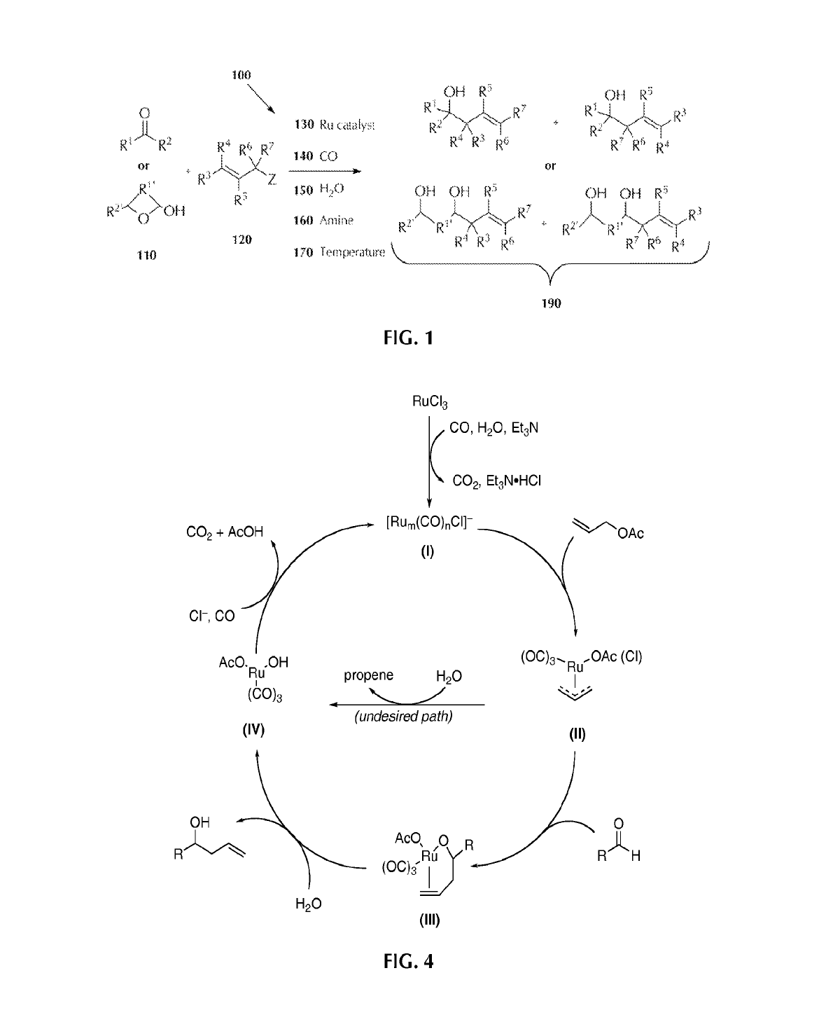

Referring to FIG. 1, method 100 includes reacting a substrate 110 and an allyl donor 120 in a reaction mixture, and forming a homoallylic alcohol 190 in the reaction mixture. The reaction mixture may include a ruthenium catalyst 130, a halide, carbon monoxide 140 at a level of at least 1 equivalent relative to the substrate 110, water 150 at a level of at least 1 equivalent relative to the substrate 110, and an amine 160 at a level of from 0.01 to 0.5 equivalent relative to the substrate 110. Alternatively, the reaction mixture may include a halide-free ruthenium catalyst 130, carbon monoxide 140 at a level of at least 1 equivalent relative to the substrate 110, water 150 at a level of at least 1 equivalent relative to the substrate 110, and an amine 160 at a level of from 0 to 0.5 equivalent relative to the substrate 110, where the reaction mixture does not include a halide. The reacting includes maintaining the reaction mixture at a temperature 170 of at least 40° C.

The reaction can be performed under batch conditions, for example, while maintaining control of pressure and temperature conditions of the batch mixture. Water 150 can accelerate the reaction, but also may cause consumption of the allyl donor 120 via an unproductive pathway. Further, when the amount of Et₃N is reduced to 0.1 equivalent or less and the water is adjusted to 1-1.5 equivalents, the reaction can proceed to 95% conversion at 70° C. in 24 h.

R¹ and R¹′ in the substrate 110 independently may be an organic group. Preferably R¹′ is an organic group having at least 2 carbon atoms between the carbon bonded to R²′ and the carbon bonded to the —OH group. R² and R²′ independently may be an organic group or hydrogen, and preferably are hydrogen. Preferably, the substrate 110 is an aldehyde or a cyclic hemiacetal.

The substrate 110 may be an aldehyde. For aldehyde substrates, R¹ may be an alkyl group, a heteroalkyl group, an alkenyl group, a heteroalkenyl group, an aryl group, or a heterocyclic group. R¹ of the substrate 110 can include but is not limited to linear or branched alkyl groups as well as ring structures either alone or conjugated, alkenyl groups, aromatic and heteroaromatic groups that may or may not be substituted with N, O, and/or S elements. The substrate 110 can also be a polyhydroxylated aldehyde such as glucose, ribose or other carbohydrate.

Example aldehydes include benzaldehyde, 4-methoxybenzaldehyde, 3-methoxybenzaldehyde, 2-methoxybenzaldehyde, 4-dimethylaminobenzaldehyde, 2-hydroxybenzaldehyde, 2-bromobenzaldehyde, 4-methyl benzaldehyde, 2-methylbenzaldehyde, 2,4,6-trimethylbenzaldehyde, 1-naphthylaldehyde, 2-furaldehyde, 2-thiophenecarboxaldehyde, N-tosyl-pyrrole-2-carboxaldehyde, 4-(trifluoromethyl)benzaldehyde, 4-nitrobenzaldehyde, 3-nitrobenzaldehyde, 2-nitrobenzaldehyde, methyl-4-formyl-benzoate, cinnamaldehyde, α-methyl-E-cinnamaldehyde, 1-cyclohexene-1-carboxaldehyde, hexanal, hydrocinnamaldehyde, cyclohexanecarboxaldehyde, pivaldehyde, (D)-glyceraldehyde acetonide. In accordance with example implementations, the substrate 110 can be benzaldehyde.

The substrate 110 may be a cyclic hemiacetal. For cyclic hemiacetal substrates, R^{1″} may be an alkyl group, a heteroalkyl group, an alkenyl group, a heteroalkenyl group, an aryl group, or a heterocyclic group. R^{1′} of the substrate 110 can include but is not limited to linear or branched alkyl groups as well as ring structures either alone or conjugated, alkenyl groups, aromatic and heteroaromatic groups that may or may not be substituted with N, O, and/or S elements. The substrate 110 can also be a polyhydroxylated aldehyde such as glucose, ribose or other carbohydrate in cyclic form. Example cyclic hemiacetals include tetrahydro-2H-pyran-2-ol and tetrahydrofuran-2-ol.

According to example implementations, the substrate 110 can be considered the limiting reagent. The amounts of all other reagents utilized in the method can be given in terms of equivalents and/or mole % in relation to the substrate 110.

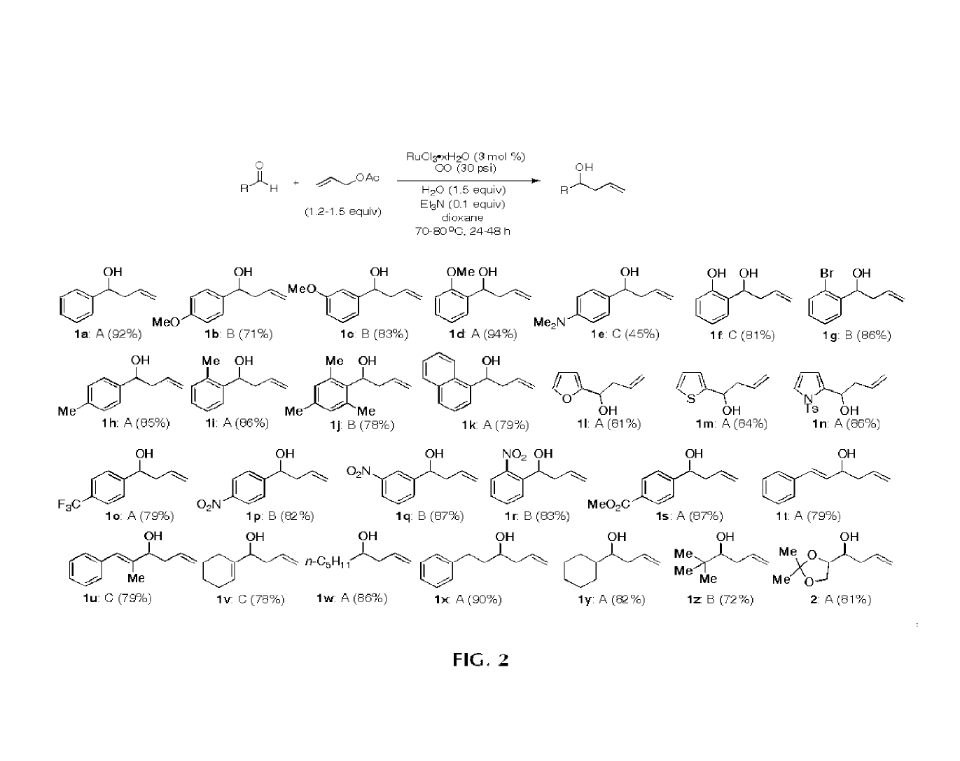

FIG. 2 represents chemical structures, reaction schemes and product yields (in parentheses) for examples of reactions of various aldehyde substrates with allyl acetate to form homoallylic alcohols. The labels “A”, “B” and “C” refer to three different sets of reaction conditions. In conditions A (1a, 1a, 1d, 1h, 1i, 1k-1o, 1s, 1t, 1a, 1w-1y, 2), the allyl acetate was present at a level of 1.2 equivalents relative to the substrate, and the reaction mixture was maintained at 70° C. for 24 hours. To achieve full conversion with slower acting aldehydes, a minor increase in temperature (conditions B) and/or an increase in reaction time and in the amount of allyl donor (conditions C) were used. In conditions B (1b, 1c, 1g, 1j, 1p-1r, 1z), the allyl acetate was present at a level of 1.2 equivalents relative to the substrate, and the reaction mixture was maintained at 80° C. for 24 hours. In conditions C (1e, 1f, 1u, 1v), the allyl acetate was present at a level of 1.5 equivalents relative to the substrate, and the reaction mixture was maintained at 80° C. for 48 hours. Experimental details are provided in Examples 1-27.

For aromatic aldehydes, such as those listed in FIG. 2, the reaction may be insensitive to electronic effects and steric effects. Both electron rich substrates (corresponding to products 1b, 1c, 1d, 1h) and electron poor substrates (corresponding to products 1o, 1p, 1q, 1r, 1s) may react with little influence of substituent location. Only the most electron rich substrate tested, with a 4-dimethylamino group (corresponding to product 1e), did not react completely under conditions C. Functional group compatibility can be quite good considering the reducing conditions. Thus, nitro, ester, hydroxyl groups (corresponding to products 1f) and a bromide (corresponding to product 1g) may be compatible. Sterically hindered aldehydes (corresponding to products 1i, 1j, 1k, 1z) can react as well. Heterocyclic aldehydes (corresponding to products 1l, 1m, 1n) reacted under the standard conditions to give good yields of the homoallylic alcohol. Olefinic aldehydes (corresponding to products 1t, 1u, 1v) reacted without problem. Linear aldehyde (corresponding to products 1w, 1x), branched aldehyde (corresponding to product 1y), and even the hindered pivalaldehyde (corresponding to product 1z) were reacted in sufficient yields. Glyceraldehyde acetonide (corresponding to product 2) reacted under the standard conditions (dr, 1.6:1) illustrating the compatibility of heteroatom-substituted substrates.

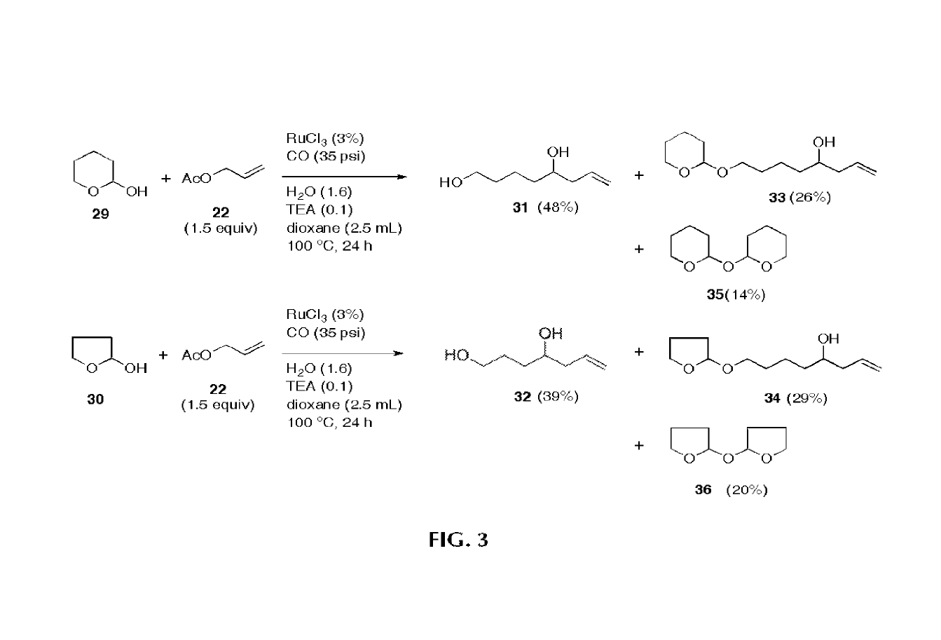

FIG. 3 represents chemical structures, reaction schemes and product yields (in parentheses) for examples of reactions of hemiacetal substrates tetrahydro-2H-pyran-2-ol (29) and tetrahydrofuran-2-ol (30) with allyl acetate (22) to form homoallylic alcohols 31 and 32, respectively. In these reactions, the amount of water was increased to 1.6 equivalents, and the reaction temperature was increased to 100° C. Homoallylic alcohol product 31 was provided in an improved yield of 60% when the amounts of water and allyl acetate were increased to 3 equivalents.

The allyl donor 120 can be considered an allyl source bearing a multitude of substituents R³-R⁷ and Z as shown in structural formula (II). The R substituents of the allyl donor 120 can be alkyl groups such as linear or branched alkyl groups as well as ring structures either alone or conjugated, alkenyl groups, aromatic and heteroaromatic groups that may or may not be substituted with N, O, and/or S elements. In accordance with specific implementations, any one or all of the R³-R⁷ substituents can be hydrogen. The groups R³-R⁷ may also be contained in rings. The substituent −Z of the allyl donor 120 can be a halide, hydroxyl, carboxyl, carbonate, carbamate, sulfate, sulfonate, phosphate, phosphonate or epoxide for example. Specific examples of allyl donor 120 include allyl acetate, vinyl oxirane, allyl alcohol, diallyl carbonate, allyl formate, α,γ-disubstituted allyl acetate, γ,γ-disubstituted allyl acetate, β-substituted allyl acetate, cinnamyl esters, crotyl esters, and 1-methylallyl acetate. Preferably −Z is an electron withdrawing organic group. Methods can provide for about 1.0 to about 1.5 equivalents of the allyl donor 120, and in other embodiments from 1.1 or 1.2 to 1.5 equivalents of the allyl donor 120.

The allyl donor 120 may be diallyl carbonate. Reaction of 1.2 equivalents of allyl carbonate (C(═O)(OCH₂CH═CH₂)₂) with benzaldehyde under the conditions of Example 1 provided 100% yield of homoallylic alcohol 1a.

The allyl donor 120 may be allyl formate. Table 1 below lists a product yield of 1a of 87% under the conditions of Example 1. This yield was increased when additives such as Pt/C and Al₂O₃ were present in the reaction mixture.

TABLE 1
Formation of Homoallylic Alcohol 1a Using Allyl Formate

| Additives | none | Pd/C (1%) | Pt/C (1%) | TiO₂ (20%) | Al₂O₃ (20%) |
|---|---|---|---|---|---|
| Product (%) | 87 | 86 | 95 | 84 | 95 |
| PhCHO (%) | 8 | 12 | 11 | 9 | 9 |
| HCOOH (%) | 74 | 85 | 88 | 78 | 83 |

The allyl donor 120 may be allyl alcohol. Reaction of allyl alcohol with benzaldehyde under the conditions of Example 1 was inefficient (20% conversion over 2 days). However, when boric anhydride was added, the reaction afforded good yield of the desired product. The yield was further improved by the addition of 0.3 equivalents of the inexpensive reagent B₂O₃ and by using 3 equivalents of allyl alcohol. Addition of 1.5 equiv of water slowed down the reaction but did not prevent it from completion. Table 2 below lists reaction conditions and product yields of 1a for reactions using allyl alcohol as the allyl donor.

TABLE 2
Formation of Homoallylic Alcohol 1a Using Allyl Alcohol

| | | | | | | |
|---|---|---|---|---|---|---|
| AllylOH (eq) | 3 | 1.5 | 3 | 3 | 3 | 2 |
| B₂O₃ (eq) | 0.3 | 0.3 | 0.3 | 0.2 | 0.1 | 0.3 |
| Temperature (° C.) | 100 | 100 | 80 | 80 | 80 | 80 |
| Time (h) | 20 | 20 | 40 | 40 | 40 | 40 |
| CO pressure (psi) | 200 | 200 | 60 | 60 | 60 | 60 |
| Product (%) | 97 | 58 | 91 | 74 | 33 | 68 |

The allyl donor 120 may be an α,γ-disubstituted allyl acetate, γ,γ-disubstituted allyl acetate or β-substituted allyl acetate. Examples 28-30 and 33 provide experimental details and results for such reactions. The allyl donor 120 may be a cinnamyl ester. Table 3 and its reaction scheme below list reaction conditions and product yields of γ-anti-9 and α-E-9 for such reactions, and Example 32 provides experimental details.

TABLE 3
Formation of Homoallylic Alcohols Using Cinnamyl Esters

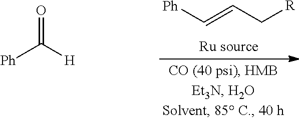

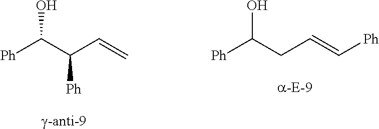 [SMILES: C=C[C@@H](c1ccccc1)[C@@H](O)c1ccccc1 ; OC(C/C=C/c1ccccc1)c1ccccc1]

| | | | Yield | |
|---|---|---|---|---|
| Ru | R | Solvent | (%) | γ:α |
| RuCl₃ | OAc | Dioxane | 78 | 1.5:1 |
| RuCl₃ | OBz | Dioxane | 16 | 1.3:1 |
| RuCl₃ | OCO₂Et | Dioxane | 82 | 3.1:1 |
| Ru₃(CO)₁₂/ | OAc | Dioxane | 37 | 1:1.3 |
| TBACl | | | | |
| RuCl₃ | OAc | EtOH | 97 | 96:1 |

The allyl donor 120 may be a crotyl ester. Table 4 and its reaction scheme below list reaction conditions and product yields of γ-anti-10 for such reactions, and Example 33 provides experimental details.

TABLE 4
Formation of Homoallylic Alcohol Using Crotyl Esters

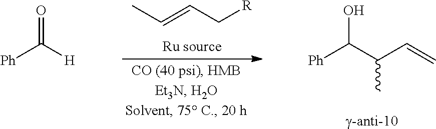

| | | | Yield | |
|---|---|---|---|---|
| Ru | R | Solvent | (%) | anti:syn |
| RuCl₃ | OAc | Dioxane | 42 | 1.6:1 |
| RuCl₃ | OCO₂Et | Dioxane | 79 | 1:1.1 |
| Ru₃(CO)₁₂/ | OBz | Dioxane | 78 | 1.8:1 |
| TBACl | | | | |
| RuCl₃ | QAc | EtOH | 83 | 1:2.8 |

The allyl donor 120 may be vinyl oxirane. Table 5 and its reaction scheme below list reaction conditions and product yields of γ-8 and α-8 for such reactions, and Example 31 provides experimental details.

TABLE 5
Formation of Homoallylic Alcohols Using Vinyl Oxirane

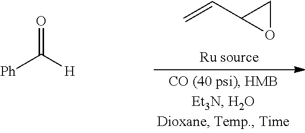

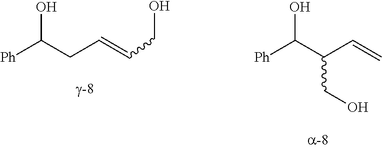

| | Vinyl- | | | γ- | | | |
|---|---|---|---|---|---|---|---|
| | oxirane | Temp. | Time | Yield | | α-Yield | |
| Ru | (eq.) | (° C.) | (h) | (%) | E:Z | (%) | anti:syn |
| RuCl₃ | 1.2 | 75 | 20 | 49 | 16:1 | 12 | 3.0:1 |
| RuCl₃ | 2.4 | 85 | 40 | 90 | 10:1 | 7 | 2.6:1 |
| Ru₃(CO)₁₂/ | 1.2 | 75 | 20 | 0 | — | 0 | — |
| TBAP | | | | | | | |
| Ru₃(CO)₁₂/ | 1.2 | 75 | 20 | 90 | 22:1 | 0 | — |
| TBACl | | | | | | | |
| Ru₃(CO)₁₂/ | 2.0 | 75 | 20 | 96 | 23:1 | 0 | — |
| TBACl | | | | | | | |

The ruthenium catalyst 130 can be provided at 0.03 or fewer equivalents of ruthenium relative to the substrate, although larger amounts may be beneficial. According to example implementations Ru-catalyst can be provided at from about 0.01 to about 0.03 equivalents of ruthenium to the substrate. The ruthenium catalyst 130 may be any ruthenium-containing substance in which the ruthenium can be reduced by carbon monoxide (CO).

The ruthenium catalyst 130 may include the halide and also may include one or more additional ligands. The Ru-catalyst can be a Ru—X-catalyst and/or a halide-free Ru-catalyst. The —X of the Ru—X-catalyst can include but is not limited to —Cl and —Br. Examples Ru—X-catalysts include but are not limited to RuCl₃, [Cp*RuCl₂]ₙ, [(COD)RuCl₂]ₙ, and [Ru(CO)₃Cl₂]₂. This Ru—X-catalyst may be provided in its hydrated form such as RuCl₃.xH₂O, for example. Under the conditions of Example 1 but with 140 psi of CO, increasing the level of RuCl₃.xH₂O from 1 mol %, to 2 mol %, and to 3 mol % provided product yields of 1a of 78%, 95% and 100%, respectively. A Ru—X-catalyst may be provided in the form of an allylmetallic catalyst. An example of an allylmetallic Ru—X-catalyst includes but is not limited to allylRu(CO)₃Br. A Ru—X-catalyst may be provided with one or more additional ligands, such as CO, cyclopentadienyl (Cp) or cyclooctadiene (COD).

The ruthenium catalyst 130 may be a halide-free catalyst. Example halide-free Ru-catalysts include but are not limited to allylRu(CO)₃OAc, Ru₃(CO)₁₂, allylRu(CO)₃OBz, (Et₄N)₂[Ru₆C(CO)₁₆]. A reaction mixture that includes a halide-free ruthenium catalyst may include no halide. Such a halide-free reaction mixture may also include no amine 160, or it may include an amine 160 at a level of less than 0.5 equivalents relative to the substrate 110.

A reaction mixture that includes a halide-free ruthenium catalyst 130 may include a halide-containing substance. The method can include providing one or both of a soluble halide or carboxylate salts as halide additives. The halide salt can be tetrabutylammonium chloride and the carboxylate salt can include tetrabutylammonium acetate. At least about 0.01 equivalents of halide supplement to the substrate may be utilized.

Table 6 lists reaction conditions and product yields of 1a from the reaction of benzaldehyde with allyl acetate under the conditions of Example 1, using 140 psi of CO, and using only the amount of amine and/or halide additives listed for each entry.

TABLE 6
Formation of Homoallylic Alcohol 1a Using Various Ru-Catalysts

| | | Et₃N | TBACl | |
|---|---|---|---|---|
| entry | Ru sources | (equiv) | (equiv) | Product % |
| 1 | RuCl₃•xH₂O | 0 | 0 | 0 |
| 2 | RuCl₃•xH₂O | 0.1 | 0 | 95 |
| 3 | allylRu(CO)₃Br | 0 | 0 | 12 |
| 4 | allylRu(CO)₃Br | 0.1 | 0 | 93 |
| 5 | allylRu(CO)₃OAc | 0 | 0 | 43 |
| 6 | allylRu(CO)₃OAc | 0.1 | 0 | 70 |
| 7 | allylRu(CO)₃OAc | 0 | 0.03 | 84 |
| 8 | Ru₃(CO)₁₂^{a} | 0 | 0 | 15 |
| 9 | Ru₃(CO)₁₂^{a} | 0 | 0.03 | 78 |

^{a}1 mol % was used

The observation that reactions with RuCl₃ and allylRu(CO)₃Br showed higher conversions than reactions with allylRu(CO)₃OAc (Table 6, entries 2, 4, 6) may indicate a possible halide affect. Indeed, when tetrabutylammonium chloride (TBACl) was added to a reaction catalyzed by allylRu(CO)₃OAc, the conversion was improved from 43 to 84% (Table 6, entries 5 and 7). The conversion was improved from 15 to 78% when Ru₃(CO)₁₂ was utilized as the precatalyst (Table 6, entries 8 and 9).

Tables 7, 8 and 9 list reaction conditions and product yields of 1a for the reaction of benzaldehyde with allyl acetate, using the salt additive listed for each entry. In Table 7, the catalyst was 3 mol % Ru₃(CO)₁₂, and the reaction was performed at 70° C. for 18 hours.

TABLE 7
Formation of Homoallylic Alcohol 1a Using Ru₃(CO)₁₂ Catalyst

| | Salt (equiv) | | | | | | | | | |
|---|---|---|---|---|---|---|---|---|---|---|
| | TBACl | | | | | TBAOAc | LiCl | LiOAc | TBAI | |
| | 0.00 | 0.03 | 0.10 | 0.20 | 0.05 | 0.10 | 0.50 | 0.10 | 0.03 | 0.10 |
| Product (%) | 15 | 78 | 80 | 75 | 65 | 49 | 43 | 28 | 64 | 68 |
| AllylOAc (%) | 95 | 0 | 0 | 0 | 0 | 0 | 59 | 75 | 27 | 22 |
| Propene (%) | 0 | 5 | 5 | 7 | 7 | 20 | 0 | 0 | 10 | 15 |

In Table 8, the catalyst was 0.25 mol % Ru₃(CO)₁₂, the salt additives was 0.75 mol %, allyl acetate was present at a level of 10 equivalents, CO was present at a level of 5 equivalents (350 psi), water was present at a level of 8 equivalents, and the reaction was performed at 75° C. for 20 hours. The halide salt listed was present at a level of 0.75% relative to the substrate benzaldehyde.

TABLE 8
Formation of Homoallylic Alcohol 1a Using Ru₃(CO)₁₂ Catalyst

| Salt additives | TBACl | TBABr | PPNCl | 1-NaphBu₃NBr | BnEt₃NCl | BnBu₃NBr |
|---|---|---|---|---|---|---|
| Product ^{a} | 93 | 90 | 98 | 100 | 53 | 80 |
| AllylOAc ^{a} | 614 | 634 | 669 | 647 | 726 | 668 |
| propene ^{b} | 293 | 276 | 233 | 353 | 221 | 252 |

^{a} percentages calculated based on ¹H NMR spectra of reaction mixture
^{b} calculated by the formula: [propene] = 1000 − [product] − [AllylOAc]

In Table 9, the catalyst was 0.25 mol % Ru₃(CO)₁₂, allyl acetate was present at a level of 10 equivalents, CO was present at a level of 5 equivalents (350 psi), water was present at a level of 8 equivalents, and the reaction was performed at 75° C. for 9.5 hours. The halide salt listed was present at a level of 0.75% relative to the substrate benzaldehyde.

TABLE 9
Formation of Homoallylic Alcohol 1a Using Ru₃(CO)₁₂ Catalyst

| | Salt additives | | | | | | |
|---|---|---|---|---|---|---|---|
| | TBAF | TBACl | TBABr | TBAI | TBAOAc | (TBA)₂HPO₄ | TBAHSO₄ |
| Product ^{a} | 59 | 68 | 77 | 80 | 37 | 47 | 25 |
| AllylOAc ^{a} | 686 | 670 | 637 | 686 | 711 | 727 | 693 |
| propene ^{b} | 255 | 262 | 286 | 234 | 252 | 226 | 282 |

| | Salt additives | | | | |
|---|---|---|---|---|---|
| | PPNCl | BnBu₃NBr | BnEt₃NCl | CetylMe₃NBr | CetylBnMe₂NCl |
| Product ^{a} | 62 | 43 | 16 | 31 | 22 |
| AllylOAc ^{a} | 730 | 838 | 907 | 924 | 877 |
| propene ^{b} | 208 | 119 | 77 | 45 | 101 |

| | Salt additives | | | | |
|---|---|---|---|---|---|
| | 1-NaphBu₃NX | | 2-NaphBu₃NX | | |
| | X = Br | X = I | X = Br | X = I | 9-AnthBu₃NBr |
| Product ^{a} | 61 | 71 | 69 | 72 | 38 |
| AlIylOAc ^{a} | 781 | 773 | 768 | 770 | 739 |
| propene ^{b} | 158 | 156 | 163 | 158 | 223 |

^{a} percentages calculated based on ¹H NMR spectra of reaction mixture
^{b} calculated by the formula: [propene] = 1000 − [product] − [AllylOAc]

FIG. 4 represents a possible reaction pathway for the reaction of an aldehyde substrate with allyl acetate. Without being held to any particular theory, the rate enhancement caused by chloride observed in this process may be due to the chloride-ligated anionic complexes (I) formed by displacement of carbon monoxide ligand(s) from the neutral Ru(0) species by chloride. These anionic complexes may be more nucleophilic than the neutral Ru(0) complexes and thus may readily react with allyl acetate to form the requisite π-allyl-Ru complex (II) that can deliver the allyl group to the aldehyde. This mechanistic formulation can provide an insight into the roles of CO and water, i.e., that the combination of CO and water may provide the stoichiometric reducing equivalents (water gas shift reaction). A plausible mechanism for the catalyst turnover can involve hydrolysis of the ruthenium alkoxide complex (III) to release the homoallylic alcohol and generate a ruthenium hydroxide species (IV). Insertion of CO into the Ru—OH bond of complex IV, followed by extrusion of CO₂ and subsequent reductive elimination of acetic acid regenerates the Ru(0) catalyst.

The carbon monoxide 140 is present in the reaction mixture at a level of at least 1 equivalent relative to the substrate. In accordance with example implementations, CO may be provided at greater than 1.0 equivalents to the substrate. CO may be added in the form of a gas as a part of the headspace above the reaction mixture. The CO can be provided to the mixture at atmospheric or superatmospheric pressures as high as 3 atmospheres. In accordance with example embodiments, less than or equal to about 35 psi CO can be provided to the reaction mixture. The CO pressure may also be maintained between from about 14 psi to about 35 psi.

The method can be performed at pressures from 15 psi to 200 psi of CO and the conversions may not substantially change at pressures above 30 psi. The reactions may proceed even at 15 psi of CO but can require longer time. The CO pressure can be kept at 30-35 psi which can be considered a substantial improvement over the prior art for at least the reason that the method can now be adapted to conventional reactors.

Table 10 lists reaction conditions and product yields of 1a for the reaction of benzaldehyde with allyl acetate under the conditions of Example 1 for 18 hours, using the pressures of CO listed.

TABLE 10
Formation of Homoallylic Alcohol 1a Using Various CO Pressures

| CO pressure (psi) | 15 | 30 | 60 | 90 | 120 | 160 | 200 |
|---|---|---|---|---|---|---|---|
| Product (%) | 48 | 95 | 95 | 100 | 98 | 95 | 95 |
| Allyl acetate (%) | 56 | 0 | 3 | 0 | 0 | 0 | 0 |
| Propene (%) | 0 | 6 | 5 | 5 | 3 | 7 | 5 |

Table 11 lists reaction conditions and product yields of 1a for the reaction of benzaldehyde with allyl acetate under the conditions of Example 1 for 8 hours, using the pressures of CO listed.

TABLE 11
Formation of Homoallylic Alcohol 1a Using Various CO Pressures

| | CO pressure (psi) | | |
|---|---|---|---|
| | 30 | 160 | 250 |
| Product (%) | 74 | 70 | 70 |
| Allyl acetate (%) | 35 | 23 | 35 |

The water 150 is present in the reaction mixture at a level of at least 1 equivalent relative to the substrate. In accordance with example implementations, water can be provided at about 1.5 equivalents to the substrate. Preferably the water is present at a level of 1 to 2 equivalents relative to the substrate. More preferably the water is present at a level of 1 to 1.5 equivalents relative to the substrate. Excess of water can shorten reaction time, provided that there is enough of the allyl donor in the reaction mixture. One possible reason for this result is that an excess of the allyl donor may compensate for side reactions such as formation of propene (see FIG. 4).

In contrast, Table 12 lists reaction conditions and product yields of 1a for the reaction of benzaldehyde with allyl acetate under conventional allylation conditions, such as those reported in Tsuji, Y.; Mukai, T.; Kondo, T.; Watanabe, Y. J. Organometallic Chem. 1989, 369, C51-053. The percentages of allyl acetate and product 1a were calculated based on ¹H NMR integration of the reaction mixture, compared to that of the inert internal standard hexamethylbenzene (HMB). Additions of water completely inhibited formation of the desired homoallylic product.

TABLE 12
Formation of Homoallylic Alcohol 1a Using Conventional Allylation
Conditions And Various Concentrations of Water

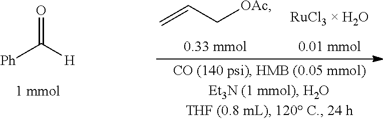

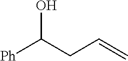

| | H₂O | allyl acetate | product |
|---|---|---|---|
| Entry | (mmol) | (%) | (%) |
| 1 | 0 | 0 | 73 |
| 2 | 0.5 | 0 | 0 |
| 3 | 1.0 | 0 | 0 |
| 4 | 1.5 | 0 | 0 |

The amine 160 can be, but is not limited to, a mono-, di-, and/or tri-substituted amine and/or a non-reducing amine such as a mono-, di-, and/or tri-alkylamine. For example, the amine can be triethylamine (Et₃N). In accordance with this implementation, the amine 160, for example, Et₃N is not the stoichiometric reducing reagent as reported in the prior art. The Et₃N may not be considered a hydride donor in this system for at least the reasons that: (i) ¹H-NMR analysis of the reaction mixtures indicated that triethylamine was not consumed; and (ii) when Et₃N was replaced by quinuclidine, an amine that cannot function as a hydride donor, the reaction still proceeded to comparable conversion in the same reaction period. In addition to Et₃N, other secondary and tertiary amines such as i-Pr₂EtN and i-Pr₂NH were also effective. However, in the absence of an amine, no reaction occurred when RuCl₃.xH₂O was used as catalyst (Table 6 above, entries 1, 2).

The amine 160, when used in the reaction, is preferably present in the reaction mixture at a level of from 0.01 to 0.5 equivalent relative to the substrate. Preferably the amine is present at a level of from 0.1 to 0.5 equivalent relative to the substrate. When a halide is present in the reaction, either as the −X in a Ru—X-catalyst or as a separate substance, the amine may be provided at a level similar to that of the halide. Preferably, the number of equivalents of amine is within 30% of the number of equivalents of halide in the reaction mixture. More preferably, the number of equivalents of amine is within 20% of the number of equivalents of halide in the reaction mixture, preferably is within 10% of the number of equivalents of halide in the reaction mixture, and preferably is within 5% of the number of equivalents of halide in the reaction mixture. Reactions using preformed Ru-allyl complexes or Ru complexes of zero valent Ru as catalyst provided homoallylic alcohol product without addition of an amine.

AllylRu(CO)₃Br and/or allylRu(CO)₃OAc can be prepared and used in the catalytic reactions as shown in Table 6. Addition of Et₃N in both cases can improve the conversions. The data of Table 6 can suggest that the amine is acting as a base, perhaps to neutralize HCl generated during the reduction of RuCl₃ (or HBr when allylRu(CO)₃Br is used), and to partially buffer the medium from becoming too acidic, as HX and acetic acid can be generated as byproducts. An excess of Et₃N, on the other hand, may cause the unproductive consumption of allyl acetate, possibly because Et₃N competitively binds to the allyl-Ru complex and thus inhibits the allylation process while favoring the undesired protonolysis of the π-allyl complex to produce propene (see FIG. 4).

Table 13 lists reaction conditions and product yields of 1a for the reaction of benzaldehyde with allyl acetate under the conditions of Example 1 for 24 hours, using 0.1 equivalent of one of the various amines listed.

TABLE 13
Formation of Homoallylic Alcohol 1a Using Various Amines

| Amine (0.1 equiv) | TEA | DIPEA | Py | DIPA | Quinuclidine | Quinine | Spartein | 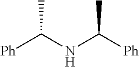 [SMILES: C[C@H](N[C@@H](C)c1ccccc1)c1ccccc1] |
|---|---|---|---|---|---|---|---|---|
| Product (% ) | 96 | 100 | 0 | 89 | 94 | 62 | 97 | 57 |
| PhCHO (%) | 3 | 0 | 100 | 11 | 3 | 38 | 2 | 42 |
| AllylOAc (%) | 0 | 0 | 110 | 0 | 0 | 36 | 0 | 47 |
| Propene (%) | 3 | 3 | 0 | 5 | 3 | 3 | 5 | 2 |

(percentages calculated based on NMR analysis of the reaction mixture)

The reaction mixture is maintained at a temperature 170 of at least 40° C. The method may be performed while maintaining the reaction mixture at a temperature less than about 100° C. during the exposing, such as less than about 80° C. Preferably the reaction mixture is maintained at a temperature of from 40° C. to 100° C., from 40° C. to 80° C., or from 70° C. to 80° C. Temperatures of 70-80° C. can be considered sufficient for the reaction to be complete within 24-48 h for most substrates. The reaction may not proceed efficiently at temperatures as low as 40° C., or it may require longer reaction times.

The reaction mixture also may include a solvent. Examples of solvents include dioxane, tetrahydrofuran, tert-butanol, iso-propanol, ethanol, ethyl acetate, acetone, cyclohexanone, N,N-dimethylformamide, dimethyl sulfoxide. Preferably the reaction mixture includes a solvent such as tetrahydrofuran, tert-butanol, ethyl acetate or cyclohexanone. Selection of the solvent can be dependent on the particular substrate and the allyl donor, and a mixed solvent may be used.

Table 14 lists reaction conditions and product yields of 1a for the reaction of benzaldehyde with allyl acetate under the conditions of Example 1 for 20 hours, using the solvent listed.

TABLE 14
Formation of Homoallylic Alcohol 1a Using A Solvent

| Solvent | AllylOAc | EtOAc | THF | DMF | DMSO | Acetone |
|---|---|---|---|---|---|---|
| Product (%) | 91 | 94 | 100 | 49 | 53 | 68 |
| AllylOAc (%) | N/A | 18 | 0 | 44 | 39 | 25 |

| Solvent | Cyclohexanone | MeOH | EtOH | i-PrOH | t-BuOH |
|---|---|---|---|---|---|
| Product (%) | 90 | 10 | 50 | 100 | 80 |
| AllylOAc (%) | 0 | 50 | 7 | <5 | 12 |
| Acetal (%) | — | 74 | 28 | — | — |

A new ruthenium-catalyzed allylation method that in accordance with particular implementations is operationally-simple, and/or highly-efficient, can utilize inexpensive and non-corrosive allyl acetate, water and a low pressure of carbon monoxide. Disclosed embodiments of the method can generate by-products that include carbon dioxide and acetic acid and as such the method can be considered environmentally benign and readily adaptable to large-scale operation. Details regarding a scale-up of the preparation of homoallylic alcohols γ-8 and 1d are provided in Examples 34 and 35, respectively. The method may utilize a vast number of substrates, be compatible with many functional groups, and/or be tolerant to electronic and steric factors.

The following examples are provided to illustrate one or more preferred embodiments of the invention. Numerous variations can be made to the following examples that lie within the scope of the invention.

### EXAMPLES

General Procedures.

All reactions were performed in a six-well autoclave equipped with a temperature probe connected to a magnetic stirrer (IKA Labortechnik) bearing a heat control element. The autoclave allowed for independent control of gas pressure in each well via the individual valves.

Materials.

Ruthenium (III) chloride hydrate was purchased from Strem Chemicals. Carbon monoxide gas (CP grade) was purchased from Si Smith Company. Dioxane (Fisher, HPLC grade) was distilled from sodium and benzophenone. Triethylamine (Aldrich, ACS grade) was distilled from CaH₂. Solvents for chromatography were: hexanes (Fisher, ACS Grade), pentane (Fisher, ACS grade), ethyl acetate (Aldrich, ACS Grade), diethyl ether (Fisher, ACS Grade), dichloromethane (Aldrich, ACS Grade), methyl tert-butyl ether (Aldrich, ACS grade).

Benzaldehyde (Aldrich), 4-methoxybenzaldehyde (Aldrich), 3-methoxy-benzaldehyde (Aldrich), 2-methoxybenzaldehyde (Aldrich), 2-hydroxybenzaldehyde (Aldrich), 2-bromobenzaldehyde (Alfa Aesar), 4-methylbenzaldehyde (Aldrich), 2-methylbenzaldehyde (Aldrich), 2,4,6-trimethylbenzaldehyde (Aldrich), 1-naphthaldehyde (Aldrich), 2-furaldehyde (Aldrich), 2-thiophenecarboxaldehyde (Aldrich), (E)-cinnamaldehyde (Aldrich), α-methyl-E-cinnamaldehyde (Aldrich), 1-cyclohexene-1-carboxaldehyde (Aldrich), n-hexanal (Aldrich), hydrocinnamaldehyde (Aldrich), cyclohexanecarboxaldehyde (Aldrich), trimethylacetaldehyde (Aldrich) were distilled prior to use. 4-(Trifluoromethyl)benzaldehyde (Aldrich) was opened and handled in the glove box. 4-(Dimethylamino)benzaldehyde (Aldrich), 4-nitro-benzaldehyde (Aldrich), 3-nitrobenzaldehyde (Aldrich), 2-nitrobenzaldehyde (Aldrich), methyl-4-formyl-benzoate (TCl) were sublimed prior to use.

Instrumentation.

Analytical thin-layer chromatography was performed on Merck silica gel plates with QF-254 indicator. Visualization was accomplished with UV (254 nm), iodine, ceric ammonium molybdate (CAM) staining solution. Column chromatography was performed using Merck silica gel (grade 9385, mesh 230-400).

¹H NMR, ¹³C NMR, ¹⁹F NMR were recorded on Varian Unity 400 (400 MHz, ¹H; 100 MHz, ¹³C), Varian Inova 500 (500 MHz, ¹H), and Varian VXR 500 (499 MHz, ¹H; 125 MHz ¹³C) spectrometer. Spectra were referenced to residual chloroform (7.26 ppm, ¹H, 77.00 ppm, ¹³C). Chemical shifts are reported in ppm, multiplicities are indicated by s (singlet), d (doublet), t (triplet), q (quartet), quint (quintet), m (multiplet) and br (broad). Coupling constants, J, are reported in Hertz. The University of Illinois Mass Spectrometer Center performed Mass spectroscopy. EI and CI mass spectra were performed on a 70-VSE spectrometer. ESI mass spectra were performed on a Micromass Quattro spectrometer. Data are reported in the form of (m/z). Infrared spectra (IR) were recorded on a Mattson Galaxy 5020 spectrophotometer in NaCl cells. Peaks are reported in cm⁻¹ with indicated relative intensities: s (strong, 67-100%); m (medium, 34-66%); w (weak, 0-33%).

### Example 1

### General Procedure for Preparation of Homoallylic Alcohols

### Preparation of 1-Phenyl-3-buten-1-ol (1a)

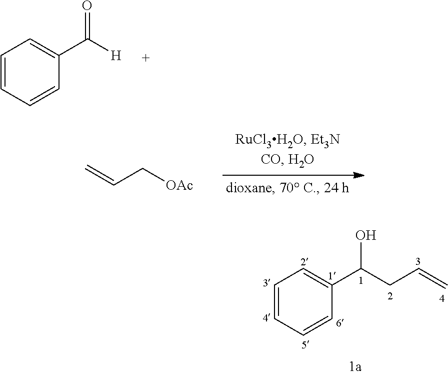

To a 10-mL flat bottom glass tube (1.5×6.5 cm) containing a Teflon-coated, magnetic stir bar were added benzaldehyde (102 μL, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL). The tube was placed in a six-well autoclave which allows six separate reactions to be conducted at the same time. The autoclave was sealed and connected to a CO gas cylinder. The autoclave was charged with CO gas (30-40 psi), the pressure was released to a vented hood four times before the CO gas was maintained at 30 psi and all the valves were closed. The autoclave was mounted onto a magnetic stirrer which was connected to a temperature probe. The probe was inserted into the metal block of the autoclave. The temperature was set at 70° C. and stirring was started. The temperature reached 70° C. within 15 min and was maintained for 24 h. The autoclave was removed from the stirrer and chilled in an ice bath. The temperature reached 10-20° C. within 40 min. The outlet was connected to a vented hood and the pressure in the autoclave was gently released. The inlet was then connected to a nitrogen line. The system was purged by N2 for 10 min before the autoclave was opened. The yellow reaction mixture containing some white precipitate of Et₃N.HCl was transferred to a 100-mL, round-bottom flask with the aid of 3 mL of diethyl ether. The solvent was removed under reduced pressure by rotary evaporation (25° C., 20 mmHg). The yellow residue was purified by silica gel column chromatography (12 g SiO₂, column size: 2.2×35 cm) eluted with hexane/MTBE (4:1, 200 mL) to provide 1a (136 mg, 92% yield) as a colorless oil.

¹H NMR: (400 MHz, CDCl₃) 7.39-7.26 (m, 5H, (Aryl)), 5.81 (ddt, J=17.2, 10.0, 6.8, 1H, HC(3)), 5.20-5.13 (m, 2H, H₂C(4)), 4.74 (ddd, J=8.1, 4.8, 3.2, 1 H, HC(1)), 2.46-2.57 (m, 2H, H₂C(2)), 2.17 (d, J=3.2, 1H, (OH)). ¹³C NMR: (100 MHz, CDCl₃) 144.0 (C(1′)), 134.4 (C(3)), 128.3 (C(3′) & C(5′)), 127.5 (C(2′) & C(6′)), 125.8 (C(4′)), 118.4 (C(4)), 73.2 (C(1)), 43.8 (C(2)). IR: (NaCl) 3390 (s), 3076 (m), 3029 (m), 2906 (m), 1640 (m), 1493 (s), 1454 (m), 1047 (s). MS: (Cl) calcd for C₁₀H₁₃O, 149.0966; found, 149.0963. TLC: R_{f} 0.23 (hexanes/MTBE, 4:1) [UV, CAM]. (Ishiyama, T.; Ahiko, T.; Miyaura, N. J. Am. Chem. Soc. 2002, 124, 12414-12415.)

### Example 2

### Preparation of 1-(4-Methoxyphenyl)-3-buten-1-ol (1b)

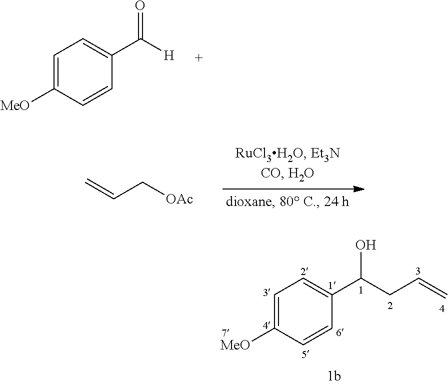

Following the procedure of Example 1, 4-methoxybenzaldehyde (136 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with hexane/MTBE (10:1, 100 mL, 4:1, 100 mL) to provide 1b (127 mg, 71%) as a colorless oil.

¹H NMR: (500 MHz, CDCl₃) 7.28 (d, J=8.8, 2H, (Aryl)), 6.89 (d, J=8.8, 2H, (Aryl)), 5.80 (ddt, J=17.3, 10.2, 7.2, 1H, CH(3)), 5.13-5.18 (m, 2H, H₂C(4)), 4.69 (t, J=6.3, 1H, HC(1)), 3.81 (s, 3H, H₃C(7′)), 2.50 (t, J=6.8, 2H, H₂C(2)), 2.15 (br, 1H, (OH)). ¹³C NMR: (125 MHz, CDCl₃) 159.0 (C(4′)), 136.0 (C(1′)), 134.6 (C(3)), 127.0 (C(3′), C(5′)), 118.2 (C(4)), 113.7 (C(2′), C(6′)), 72.9 (C(7′)), 55.2 (C(1)), 43.7 (C(2)). IR: (NaCl) 3411 (m), 3071 (m), 2934 (m), 2836 (m), 1611 (s), 1513 (s), 1465 (m), 1441 (m), 1302 (m), 1247 (s), 1175 (s). HRMS: (Cl) calcd for C₁₁H₁₅O₂, 179.1072; found, 179.1071. TLC: R_{f} 0.23 (hexanes/MTBE, 4:1) [UV, CAM]. (Ishiyama, T.; Ahiko, T.; Miyaura, N. J. Am. Chem. Soc. 2002, 124, 12414-12415.)

### Example 3

### Preparation of 1-(3-Methoxyphenyl)-3-buten-1-ol (1c)

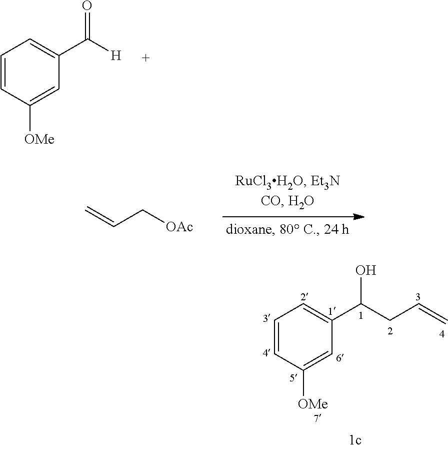

Following the General Procedure, 3-methoxybenzaldehyde (136 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with hexane/MTBE (10:1, 100 mL, 4:1, 100 mL) to provide 1c (148 mg, 83%) as a colorless oil.

¹H NMR: (500 MHz, CDCl₃) 7.29-7.26 (m, 1H, Aryl), 6.95-6.93 (m, 2H, Aryl), 6.84-6.80 (m, 1H, Ar), 5.81 (ddt, J=17.1, 10.2, 7.1, 1H, HC(3)), 5.20-5.15 (m, 2H, H₂C(4)), 4.73 (ddd, J=7.8, 4.8, 2.2, 1H, HC(1), 3.82 (s, 3H, H₃C(7′)), 2.57-2.44 (m, 2H), 2.13 (d, J=2.2, 1H, OH). ¹³C NMR: (125 MHz, CDCl₃) 159.7 (C(3′)), 145.6 (C(1′)), 134.4 (C(3)), 129.4 (C(5′)), 118.4 (C(6′)), 118.1 (C(4)), 113.0 (C(4′)), 111.3 (C(2′)), 73.2 (C(1)), 55.2 (C(7′)), 43.7 (C(2)). IR: (NaCl) 34.19 (m), 3075 (m), 3004 (m), 2937 (m), 2835 (m), 1601 (s), 1585 (s), 1489 (s), 1455 (s), 1435 (s), 1315 (m), 1287 (s), 1264 (s), 1155 (m). HRMS: (El, 70 eV) calcd for C₁₁H₁₄O₂, 178.0993; found, 178.0986. TLC: R_{f} 0.27 (hexanes/MTBE, 4:1) [UV, CAM]. (Yasuda, M.; Fujibayashi, T.; Baba, A. J. Org. Chem. 1998, 63, 6401-6404.)

### Example 4

### Preparation of 1-(2-Methoxyphenyl)-3-buten-1-ol (1d)

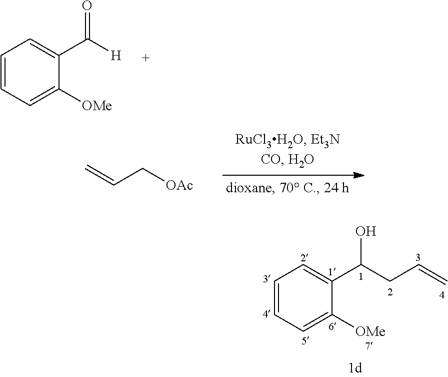

Following the procedure of Example 1, 2-methoxybenzaldehyde (136 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexane/ethyl acetate (10:1, 100 mL, 4:1, 100 mL) to provide 1d (168 mg, 94%) as a colorless oil.

¹H NMR: (500 MHz, CDCl₃) 7.37 (dd, J=7.6, 1.5, 1H, Aryl), 7.27 (td, J=8.0, 1.7, 1H, Aryl), 6.98 (td, J=8.0, 1.0, 1H, Aryl) 6.89 (d, J=8.5, 1H, Aryl), 5.85 (ddt, J=17.0, 10.2, 7.1, 1H, HC(3)), 5.14 (d, J=17.0, 1H, HₐC(4)) 5.12 (d, J=10.2, 1H, H_{b}C(4)), 4.96 (ddd, J=8.1, 5.4, 5.1, 1H, HC(1)), 3.86 (s, 3H, H₃C(7′)), 2.72 (d, J=5.4, 1H, OH), 2.63-2.47 (m, 2H, H₂C(2)). ¹³C NMR: (125 MHz, CDCl₃) 156.2 (C(2′)), 135.1 (C(3)), 131.7 (C(1′)), 128.1 (C(4′)), 126.6 (C(6′)), 120.5 (C(5′)), 117.4 (C(4)), 110.3 (C(3′)), 69.4 (C(1)), 55.1 (C(7′)), 41.7 (C(2)). IR: (NaCl) 3411 (m), 3073 (m), 2937 (m), 2836 (m), 1601 (m), 1587 (m) 1490 (s), 1464 (s), 1438 (m), 1287 (m), 1239 (s). HRMS: (ESI) calcd for C₁₁H₁₄O₂Na, 201.0891; found, 201.0894. TLC: R_{f} 0.47 (hexanes/EtOAc, 3:1) [UV, CAM]. (Yasuda, M.; Fujibayashi, T.; Baba, A. J. Org. Chem. 1998, 63, 6401-6404.)

### Example 5

### Preparation of 1-(4-(Dimethylamino)phenyl)-3-buten-1-01 (1e)

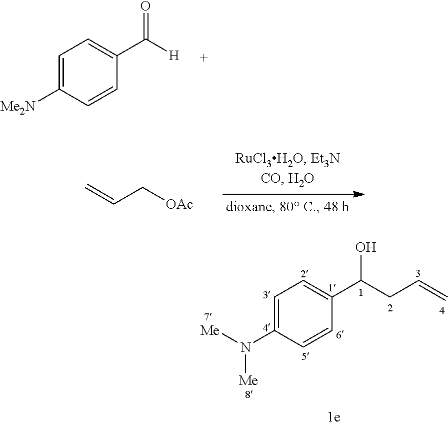

Following the procedure of Example 1, 4-dimethylaminobenzaldehyde (149 mg, 1 mmol), RuCl₃.xH₂O (6.2 mg, 0.03 mmol, 0.03 equiv), allyl acetate (163 μL, 1.5 mmol, 1.5 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 35 psi of CO at 80° C. for 48 h. Silica gel column chromatography was eluted with dichloromethane/ethyl acetate (10:0, 50 mL; 9:1, 50 mL; 4:1, 100 mL) to provide 1e (86 mg, 45%) as a pale yellow oil.

¹H NMR: (500 MHz, CDCl₃) 7.23 (d, J=8.7, 2H, Aryl), 6.73 (d, J=8.7, 2H, Aryl), 5.85-5.78 (m, 1H, HC(3)), 5.19-5.12 (m, 2H, H₂C(4)), 4.63 (td, J=6.3, 2.3, 1H, HC(1)), 2.96 (s, 6H, H₃C(7′) & H₃C(8′)), 2.56-2.50 (m, 2H, H₂C(2)), 1.95 (d, J=2.3, 1H, OH). ¹³C NMR: (125 MHz, CDCl₃) 150.2 (C(4′)), 135.0 (C(3)), 131.8 (C(1′)), 126.8 (C(2′) & C(6′)), 117.8 (C(4)), 112.5 (C(3′) & C(5′)), 73.2 (C(1)), 43.5 (C(7′) & C(8′)), 40.6 (C(2)). IR: (NaCl) 3391 (m), 3073 (m), 2966 (m), 2889 (m), 2801 (m), 1614 (s), 1567 (w), 1523 (s), 1479 (m), 1443 (m), 1348 (s), 1224 (m). HRMS: (ESI) calcd for C₁₂H₁₈NO, 192.1388; found, 192.1386. TLC: R_{f} 0.1 (dichloromethane) [UV, CAM]. (Chretien, J-M.; Zammattio, F.; Gauthier, D.; Grognec, E. L.; Paris, M.; Quintard, J-P. Chem. Eur. J. 2006, 12, 6816-6828.)

### Example 6

### Preparation of 1-(2-Hydroxy)phenyl)-3-buten-1-ol (1f)

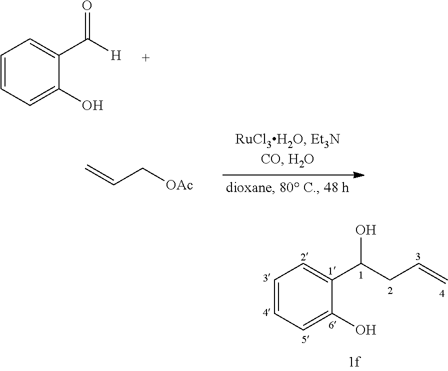

Following the procedure of Example 1, 2-hydroxybenzaldehyde (122 mg, 1 mmol), RuCl₃.xH₂O (6.2 mg, 0.03 mmol, 0.03 equiv), allyl acetate (163 μL, 1.5 mmol, 1.5 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 35 psi of CO at 80° C. for 48 h. Silica gel column chromatography was eluted with hexanes/MTBE (4:1, 300 mL) to provide 1f (132 mg, 81′)/0) as an yellow oil.

¹H NMR: (400 MHz, CDCl₃) 8.03 (br, 1H, (OH)), 7.17 (td, J=8.0, 2.5, 1 H, Aryl), 6.97 (dd, J=8.0, 2.5, 1H, Aryl), 6.89-6.82 (m, 2H, Aryl), 5.90-5.79 (m, 1H, HC(3)), 5.26-5.19 (m, 2H, H₂C(4)), 4.87 (dd, J=8.4, 5.6, 1H, HC(1)), 2.81 (br, 1H, HO), 2.68-2.54 m, 2H, H₂C(2)). ¹³C NMR: (125 MHz, CDCl₃) 155.5 (C(2′)), 133.8 (C(3)), 129.0 (C(4′)), 127.1 (C(6′)), 126.2 (C(1′)), 119.8 (C(5′)), 119.4 (C(3′)), 117.2 (C(4)), 74.6 (C(1)), 42.1 (C(2)). IR: (NaCl) 3337 (s), 3071 (s), 2976 (m), 2928 (m), 1640 (m), 1609 (m), 1587 (s), 1490 (s), 1456 (s), 1384 (m), 1349 (m), 1240 (s). HRMS: (ESI) calcd for C₁₀H₁₂O₂Na, 187.0735; found, 187.0740. TLC: R_{f} 0.32 (hexanes/EtOAc, 3/1) [UV, CAM]. (Tan, X.-L-1.; Shen, B.; Deng, W.; Zhao, H.; Liu, L.; Guo, Q.-X. Org. Let. 2003, 5, 1833-1835.)

### Example 7

### Preparation of 1-(2-Bromo)phenyl)but-3-en-1-ol (1g)

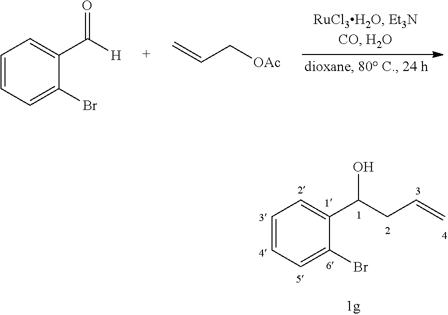

Following the procedure of Example 1, 2-bromobenzaldehyde (185 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with hexanes/MTBE (4:1, 300 mL) to provide 1g (195 mg, 86%) as a white solid.

¹H NMR: (400 MHz, CDCl₃) 7.56 (dd, J=8.0, 1.4, 1H, Aryl), 7.52 (dd, J=8.0, 1.4, 1H, Aryl), 7.34 (td, J=8.0, 1.4, 1H, Aryl), 7.13 (dt, J=8.0, 1.4, 1H, Aryl), 5.94-5.83 (m, 1H, HC(3)), 5.24-5.16 (m, 2H, H₂C(4)), 5.10 (dt, J=8.8, 3.4, 1H, HC(1)), 2.68-2.60 (m, 1H, HₐC(2)), 2.39-2.31 (m, 1H, H_{b}C(2)), 2.21 (d, J=3.4, 1H, HO). ¹³C NMR: (125 MHz, CDCl₃) 142.7 (C(1′)), 134.2 (C(3)), 132.6 (C(3′)), 128.8 (C(4′)), 127.6 (C(5′)), 127.3 (C(6′)), 121.8 (C(2′)), 118.7 (C(4)), 71.8 (C(1)), 42.1 (C(2)). IR: (NaCl) 3390 (m), 3066 (m), 2976 (m), 2905 (m), 1640 (m), 1564 (m) 1462 (s), 1434 (s), 1190 (m). HRMS: (Cl) calcd. for C₁₀H₁₀OBr, 224.9915; found, 224.9919. TLC: R_{f} 0.56 (hexanes/EtOAc, 3:1) [UV, CAM]. (Furstner, A.; Voigtlander, D. Synthesis, 2000, 7, 959-969.)

### Example 8

### Preparation of 1-p-Tolyl-3-buten-1-ol (1h)

Following the procedure of Example 1, 4-methylbenzaldehyde (120 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with pentane/MTBE (4:1, 300 mL) to provide 1h (138 mg, 85′)/0) as a colorless oil.

¹H NMR: (500 MHz, CDCl₃) 7.25 (d, J=8.1, 2H, Aryl) 7.16 (d, J=8.1, 2H, Aryl), 5.80 (ddt, J=17.3, 10.1, 7.2, 1H, HC(3)), 5.16-5.13 (m, 2H, H₂C(4)), 4.70 (td, J=5.2, 2.2, 1H, HC(1)), 2.50 (m, 2H, H₂C(2)), 2.34 (s, 3H, H₃C(7′)), 2.05 (d, J=2.2, 1H, OH). ¹³C NMR: (125 MHz, CDCl₃) 140.9 (C(1′)), 137.2 (C(3)), 134.6 (C(4′)), 129.1 (C(2′), C(6′)), 125.7 (C(3′), C(5′)), 118.2 (C(4)), 73.51 (C(1)), 43.7 (C(7′)), 21.1 (C(2)) IR: (NaCl) 3390 (m), 3071 (m), 3014 (m), 2976 (m), 2921 (s), 2857 (m), 1640 (m), 1514 (m), 1431 (m), 1306 (m), 1119 (m). HRMS: (Cl) calcd for C₁₁H₁₅O, 163.1123; found, 163.1126. TLC: R_{f} 0.23 (hexanes/MTBE, 4:1) [UV, CAM]. (Ishiyama, T.; Ahiko, T.; Miyaura, N. J. Am. Chem. Soc. 2002, 124, 12414-12415; Shen, K.-H.; Yao, C.-F, J. Org. Chem. 2006, 71, 3980-3983.)

### Example 9

### Preparation of 1-o-Tolyl-3-buten-1-ol (1i)

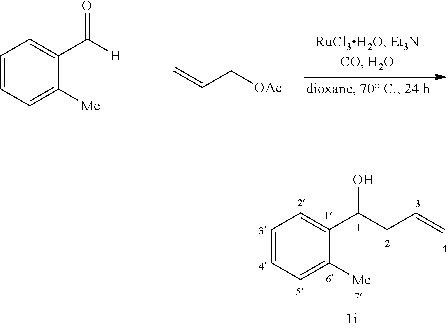

Following the procedure of Example 1, 2-methylbenzaldehyde (120 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with pentane/MTBE (4:1, 300 mL) to provide 1i (139 mg, 86′)/0) as a colorless oil.

¹H NMR: (500 MHz, CDCl₃) 7.50 (d, J=7.8, 1H, Aryl), 7.25 (t, J=7.8, 1H, Aryl), 7.19 (td, J=7.8, 1.5, 1H, Aryl), 7.14 (t, J=7.8, 1H, Aryl), 5.92-5.85 (m, 1H, HC(3)), 5.21 (d, J=17.1, 1H, HₐC(4)), 5.18 (d, J=10.0, 1H, H_{b}C(4)), 4.98 (quint, J=4.0, 1H, HC(1)), 2.55-2.41 (m, 2H, H₂C(2)), 2.36 (s, 3H, H₃C(7′)), 2.03 (d, J=4.0, 1H, HO). ¹³C NMR: (125 MHz, CDCl₃) 141.9 (C(1′)), 134.7 (C(2′)), 134.3 (C(3)), 130.3 (C(4′)), 127.2 (C(3′)), 126.2 (C(5′)), 125.1 (C(6′)), 118.2 (C(4)), 69.6 (C(1)), 42.6 (C(2)), 19.0 (C(7′)). IR: (NaCl) 3390 (m), 3074 (s), 3018 (m), 2971 (m), 2928 (m), 1640 (m), 1514 (w), 1487 (m), 1461 (s), 1434 (m), 1287 (m). HRMS: (Cl) calcd for C₁₁H₁₅O, 163.1123; found, 163.1125. TLC: R_{f} 0.23 (hexanes/MTBE, 4:1) [UV, CAM]. (Shen, K.-H.; Yao, C.-F. J. Org. Chem. 2006, 71, 3980-3983.)

### Example 10

### Preparation of 1-Mesityl-3-buten-1-ol (1j)

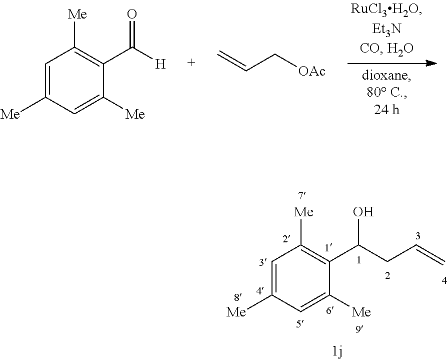

Following the procedure of Example 1, 2,4,6-trimethylbenzaldehyde (148 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with pentane/MTBE (10:1, 100 mL; 4:1, 200 mL) to provide 1j (149 mg, 78%) as a colorless oil.

¹H NMR: (500 MHz, CDCl₃) 6.82 (s, 2H, Aryl), 5.84 (dddd, J=16.8, 10.2, 7.9, 6.3, 1H, HC(3)), 5.18 (d, J=16.8, 1H, HₐC(4)), 5.17 (ddd, J=9.0, 5.1, 1.3, 1H, HC(1)), 5.12 (d, J=10.2, 1H, H_{b}C(4)), 2.71 (ddd, J=14.1, 9.0, 6.3, 1H, HₐC(2)), 2.49 (ddd, J=14.1, 7.9, 5.1, 1H, H_{b}C(2)), 2.41 (s, 6H, H₃C(7′) & H₃C(9′)), 2.25 (s, 3H, H₃C(8′)), 1.87 (d, J=1.3, 1H, HO). ¹³C NMR: (125 MHz, CDCl₃) 136.6 (C(4′)), 136.0 (C(1′)), 135.2 (C(3)), 130.1 (C(2′, 3′, 5′, 6′)), 117.7 (C(4)), 70.7 (C(1)), 40.3 (C(2)), 20.7 (C(7′, 8′, 9′)). IR: (NaCl) 3521 (m), 3390 (s), 3066 (m), 2920 (s), 2862 (m), 1639 (m), 1611 (m), 1446 (s), 1376 (m), 1306 (m), 1045 (s). HRMS: (ESI) calcd for C₁₃H₁₈ONa, 213.1255; found, 213.1261. TLC: R_{f} 0.32 (hexanes/MTBE, 4:1) [UV, CAM]. (Sumida, S.; Ohga, M.; Mitani, J.; Nokami, J. J. Am. Chem. Soc. 2000, 122, 1310-1313.)

### Example 11

### Preparation of 1-(Naphthalen-1-yl)-3-buten-1-ol (1k)

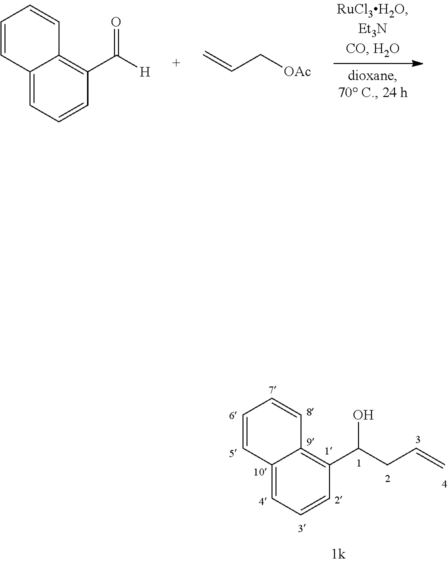

Following the procedure of Example 1, 1-naphthylaldehyde (156 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (10:1, 100 mL; 4:1, 200 mL) to provide 1k (156 mg, 79%) as a colorless oil.

¹H NMR: (500 MHz, CDCl₃) 8.08 (d, J=8.2, 1H, Aryl), 7.88 (d, J=7.8, 1H, Aryl), 7.80 (d, J=8.2, 1H, Aryl) 7.65 (d, J=7.1, 1H, Aryl), 7.55-7.45 (m, 3H, Aryl), 5.98-5.90 (m, 1H, HC(3)), 5.52 (dd, J=8.3, 4.0, 1H, HC(1)), 5.28-5.18 (m, 2H, HC(4)), 2.80-2.58 (m, 2H, H₂C(2)), 2.30 (br, 1H, HO). ¹³C NMR: (125 MHz, CDCl₃) 139.3 (Aryl), 134.7 (C(3)), 133.7 (Aryl), 130.2 (Aryl), 128.9 (Aryl), 127.9 (Aryl), 126.0 (Aryl), 125.4 (Aryl), 125.3 (Aryl), 122.9 (Aryl), 122.8 (Aryl), 118.3 (C(4)), 69.9 (C(1)), 42.8 (C(2)). IR: (NaCl) 3560 (m), 3399 (s), 3069 (m), 2928 (m), 1639 (m), 1596 (m), 1510 (m), 1431 (m), 1394 (m), 1261 (m), 1166 (m). MS: (Cl) calcd for C₁₄H₁₅O, 199.1123; found, 199.1124. TLC: R_{f} 0.61 (hexanes/EtOAc, 3:1) [UV, CAM]. (Shen, K.-H.; Yao, C.-F. J. Org. Chem. 2006, 71, 3980-3983.)

### Example 12

### Preparation of 1-(Furan-2-yl)-3-buten-1-ol (1l)

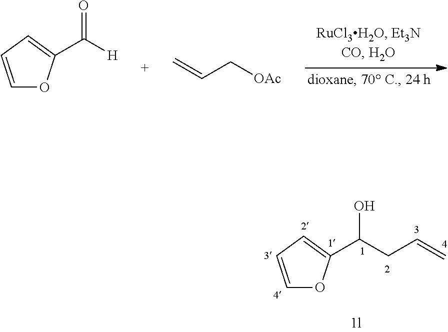

Following the procedure of Example 1, 2-furaldehyde (96 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (10:1, 100 mL; 4:1, 200 mL); solvent was removed in rotavap at 5-10° C. (25 torr) to provide 1l (112 mg, 81%) as a colorless oil.

¹H NMR: (500 MHz, CDCl₃) 7.38 (dd, J=2.0, 1.0, 1H, HC(4′)), 6.35 (d, J=3.2, 2.0, 1H, HC(3′)), 6.23 (d, J=3.5, 1.0, 1H, HC(2′)), 5.84-5.73 (m, 1H, HC(3)), 5.20-5.09 (m, 2H, H₂C(4)), 4.72 (t, J=6.5, 1H, HC(1)), 2.65-2.55 (m, 2H), 2.12 (br, 1H, OH). ¹³C NMR: (125 MHz, CDCl₃) 155.9 (C(1′)), 141.9 (C(4′)), 133.6 (C(3)), 118.5 (C(4)), 110.1, (C(2′)), 106.0 (C(3′)), 66.9 (C(1)), 40.0 (C(2)). IR: (NaCl) 3390 (s), 3075 (m), 2976 (m), 2913 (m), 1642 (m), 1506 (m), 1435 (m), 1228 (m), 1149 (s). HRMS: (Cl) calcd for C₈H₁₁O₂, 139.0759; found, 139.0760. TLC: R_{f} 0.27 (hexanes/MTBE, 4:1) [UV, CAM]. (Tan, X.-H.; Shen, B.; Deng, W.; Zhao, H.; Liu Guo, Q.-X. Org. Let. 2003, 5, 1833-1835.)

### Example 13

### Preparation of 1-(Thiophene)-3-buten-1-ol (1m)

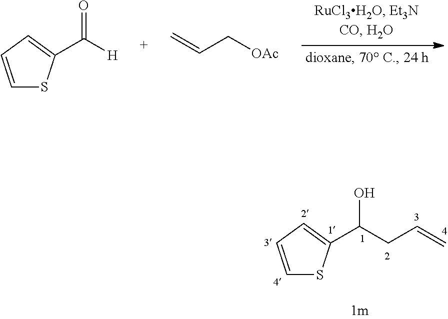

Following the procedure of Example 1, 2-thiophenecarboxaldehyde (112 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with pentane/MTBE (10:1, 100 mL; 4:1, 200 mL); solvent was removed in rotavap at 5-10° C. (25 torr) to provide 1m (127 mg, 84%) as a colorless oil.

¹H NMR: (500 MHz, CDCl₃) 7.26 (m, 1H, Aryl), 6.98 (m, 2H, Aryl), 5.88-5.77 (m, 1H, HC(3)), 5.23-5.16 (m, 2H, H₂C(4)), 5.00-4.95 (m, 1H, HC(1)), 2.65-2.59 (m, 2H, H₂C(2)), 2.32 (br, 1H, HO). ¹³C NMR: (125 MHz, CDCl₃) 147.7 (C(1′)), 133.8 (C(3)), 126.5 (C(4′)), 124.4 (C(3′)), 123.6 (C(2′)), 118.7 (C(4)), 69.3 (C(1)), 43.7 (C(2)). IR: (NaCl) 3390 (s), 3075 (m), 2978 (m), 2905 (m), 1704 (m), 1641 (m), 1435 (m), 1315 (m), 1229 (m), 1035 (s). HRMS (Cl): cacld for C₈H₁₁OS, 155.0531; found, 155.0533. TLC: R_{f} 0.29 (hexanes/MTBE, 4:1) [UV, CAM]. (Chretien, J-M.; Zammattio, F.; Gauthier, D.; Grognec, E. L.; Paris, M.; Quintard, J-P. Chem. Eur. J. 2006, 12, 6816-6828.; Shen, K.-H.; Yao, C.-F. I. Org. Chem. 2006, 71, 3980-3983.)

### Example 14

### Preparation of 1-(1-Tosyl-1H-pyrrol-2-yl)-3-buten-1-ol (1n)

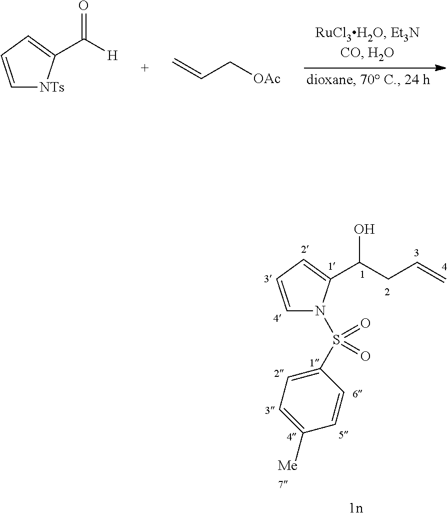

Following the procedure of Example 1, N-tosyl-pyrrole-2-carboxaldehyde (249 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (4:1, 100 mL), hexanes/ethyl acetate (3:1, 200 mL) to provide 1n (250 mg, 86%) as a colorless oil. N-tosyl-pyrrole-2-carboxaldehyde was prepared according to Masquelin, T. et al. Helv. Chim. Acta. 1994, 77, 1395-1411.

¹H NMR: (500 MHz, CDCl₃) 7.70 (d, J=8.5, 2H, Aryl), 7.32-7.30 (m, 3H, Aryl), 6.32-6.30 (m, 1H, Aryl), 6.26 (t, J=3.2, 1H, Aryl), 5.79 (ddt, J=17.3, 10.3, 6.8, 1H, HC(3)), 5.11-5.05 (m, 2H, H₂C(4)), 4.96 (td, J=6.3, 3.2, 1H, HC(1)), 2.81 (d, J=3.2, 1H, OH), 2.62-2.58 (m, 2H, H₂C(2)), 2.42 (s, 3H, H₃C(7″)). ¹³C NMR: (125 MHz, CDCl₃) 145.1 (C(1′)), 137.3 (C(1″)), 136.2 (C(4″)), 134.4 (C(3)), 130.0 (C(3″) & C(5″)), 126.6 (C(2″) & C(6″)), 123.4 (C(4′)), 117.7 (C(4)), 112.6 (C(3′)), 111.6 (C(2′)), 64.9 (C(1)), 39.8 (C(2)), 21.6 (C(7″)). IR: (NaCl) 3553 (m), 3390 (m), 3148 (m), 3073 (m), 2977 (m), 2925 (m), 1774 (s), 1640 (m), 1596 (s), 1494 (m), 1476 (m), 1452 (m), 1402 (m), 1366 (s), 1307 (m), 1291 (m), 1236 (m), 1190 (s), 1173 (s), 1151 (s), 1089 (s), 1057 (s), 1017 (s). HRMS: (ESI) calcd for C₁₅H₁₇NO₃SNa, 314.0827; found, 314.0827. TLC: R_{f} 0.25 (hexanes/ethyl acetate, 3:1) [UV, CAM]. (Zhou, W-S.; Wei, D. J. Chem. Res. 1993, 8, 290-295.)

### Example 15

### Preparation of 1-(4-(Trifluoromethyl)phenyl)-3-buten-1-ol (1o)

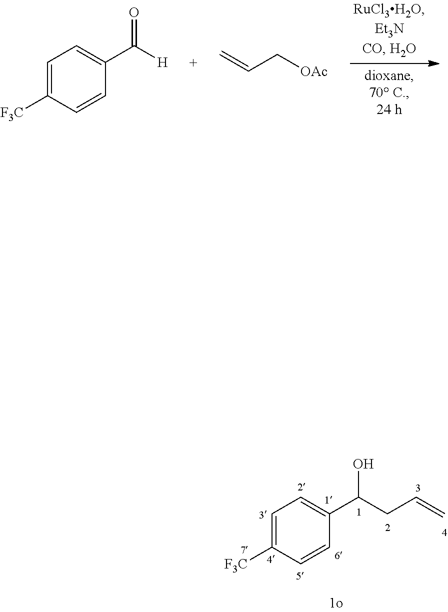

Following the procedure of Example 1,4-(trifluoromethyl)benzaldehyde (174 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (4:1, 250 mL) to provide 10 (170 mg, 79%) as a pale yellow oil.

¹H NMR: (400 MHz, CDCl₃) 7.58 (d, J=8.3, 2H, Aryl), 7.45 (d, J=8.3, 2H, Aryl), 5.83-5.72 (m, 1H, HC(3)), 5.21-5.17 (m, 2H, H₂C(4)), 4.79 (td, J=7.8, 4.0, 1H, HC(1)), 2.58-2.43 (m, 2H, H₂C(2)), 2.22 (br, 1H, HO). ¹³C NMR: (125 MHz, CDCl₃) 147.7 (C(1′)), 133.6 (C(3)), 129.7 (q, J=32.3, C(4′)), 126.1 (C(2′) & C(6′)), 125.3 (q, J=3.7, C(3′) & C(5′)), 124.1 (q, J=261.6, C(7′)), 119.1 (C(4)), 72.5 (C(1)), 43.8 (C(2)). IR: (NaCl) 3390 (s), 3079 (m), 2982 (m), 2909 (m), 1642 (m), 1621 (m), 1417 (m), 1326 (s), 1164 (s), 1125 (s), 1068 (s), 1017 (s). HRMS: (Cl) calcd for C₁₁H₁₂OF₃, 217.0840; found, 217.0846. TLC: R_{f} 0.52 (hexanes/ethyl acetate, 3:1) [UV, 12, CAM]. (Ishiyama, T.; Ahiko, T.; Miyaura, N. J. Am. Chem. Soc. 2002, 124, 12414-12415.)

### Example 16

### Preparation of 1-(4-Nitrophenyl)-3-buten-1-ol (1p)

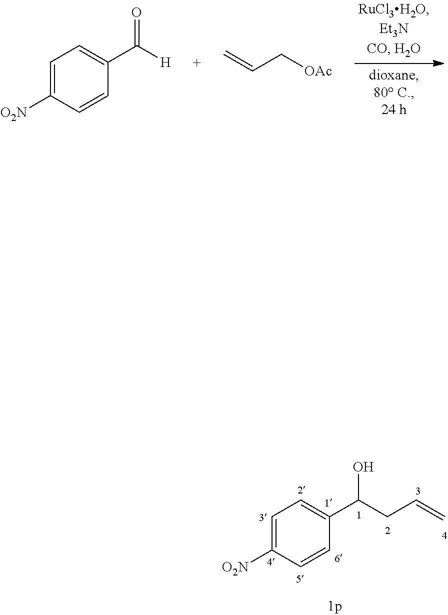

Following the procedure of Example 1, 4-nitrobenzaldehyde (151 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with hexanes/ethyl acetate (5:1, 450 mL) to provide 1p (158 mg, 82%) as a pale yellow oil.

¹H NMR: (500 MHz, CDCl₃) 8.19 (d, J=8.7, 2H, Aryl), 7.52 (d, J=8.7, 2H, Aryl), 5.78 (dddd, J=16.9, 11.2, 7.8, 6.6, 1H, HC(3)), 5.20-5.15 (m, 2H, H₂C(4)), 4.86 (quint, J=3.9, 1H, HC(1)), 2.58-2.42 (m, 2H, H₂C(2)), 2.31 (br, 1H, HO). ¹³C NMR: (125 MHz, CDCl₃) 151.1 (C(4′)), 147.2 (C(1′)), 133.2 (C(3)), 126.5 (C(3′) & C(5′)), 123.6 (C(2′) & C(6′)), 119.6 (C(4)), 72.1 (C(1)), 43.8 (C(2)). IR: (NaCl) 411 (m), 3078 (w), 2908 (w), 1641 (w), 1605 (m), 1517 (s), 1346 (s), 1108 (m), 1055 (m), 1013 (m), 920 (m), 854 (m). HRMS: (ESI) calcd for C₁₀H₁₂NO₃, 194.0817; found, 194.0820. TLC: R_{f} 0.40 (hexanes/ethyl acetate, 4:1) [UV, CAM]. (Chretien, J-M.; Zammattio, F.; Gauthier, D.; Grognec, E. L.; Paris, M.; Quintard, J-P. Chem. Eur. J. 2006, 12, 6816-6828.; Furstner, A.; Voigtlander, D. Synthesis, 2000, 7, 959-969.)

### Example 17

### Preparation of 1-(3-Nitrophenyl)-3-buten-1-ol (1q)

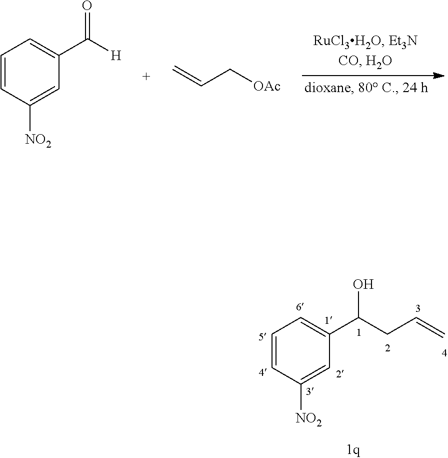

Following the procedure of Example 1, 3-nitrobenzaldehyde (151 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with hexanes/ethyl acetate (5:1, 450 mL) to provide 1q (168 mg, 87%) as a colorless oil.

¹H NMR: (500 MHz, CDCl₃) 8.22 (t, J=1.5, 1H, Aryl), 8.11 (ddd, J=8.0, 2.5, 1.0, 1H, Aryl), 7.69 (d, J=8.0, 1H, Aryl), 7.51 (t, J=8.0, 1H, Aryl), 5.79 (dddd, J=16.5, 10.0, 8.0, 7.0, 1H, HC(3)), 5.19-5.15 (m, 2H, H₂C(4)), 4.85 (quint, J=3.5, 1H, HC(1)), 2.59-2.44 (m, 2H, H₂C(2)), 2.38 (d, J=3.5, 1H, HO). ¹³C NMR: (125 MHz, CDCl₃) 148.2 (C(3′)), 145.9 (C(1′)), 133.2 (C(3)), 131.9 (C(5′)), 129.3 (C(6′)), 122.4 (C(2′)), 120.8 (C(4′)), 119.5 (C(4)), 72.0 (C(1)), 43.8 (C2)). IR: (NaCl) 3411 (m), 3077 (w), 2976 (w), 2907 (w), 1641 (m), 1529 (s), 1479 (m), 1436 (m), 1350 (s), 1200 (m), 1093 (m), 1055 (m), 922 (m), 808 (m). HRMS: (Cl) calcd for C₁₀H₁₂NO₃, 194.0817; found, 194.0815. TLC: R_{f} 0.29 (hexanes/ethyl acetate, 5:1) [UV, CAM]. (Hosomi, A.; Kohra, S.; Ogata, K.; Yanagi, T.; Tominaga, Y. I. Org. Chem. 1990, 55, 2415-2420.)

### Example 18

### Preparation of 1-(2-Nitrophenyl)-3-buten-1-ol (1r)

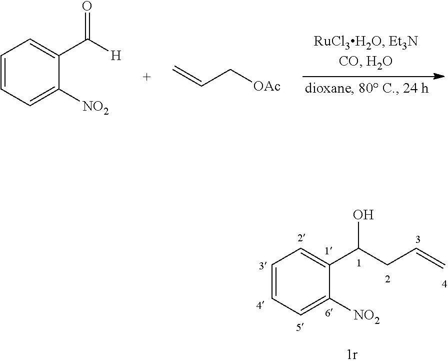

Following the procedure of Example 1, 2-nitrobenzaldehyde (151 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. Silica gel column chromatography was eluted with hexanes/MTBE (4:1, 400 mL) to provide 1r (160 mg, 83%) as a pale yellow oil.

¹H NMR: (500 MHz, CDCl₃) 7.92 (dd, J=8.0, 1.5, 1H, Aryl), 7.82 (dd, J=8.0, 2.5, 1.0, 1H, Aryl), 7.64 (td, J=8.0, 1.5, 1H, Aryl), 7.51 (td, J=8.0, 1.5, 1H, Aryl), 5.89 (dddd, J=17.0, 9.0, 7.5, 6.0, 1H, HC(3)), 5.31 (dd, J=8.5, 3.5, 1H, HC(1)), 5.22-5.17 (m, 2H, H₂C(4)), 2.72-2.67 (m, 1H, HₐC(2)), 2.50 (br, 1H, HO), 2.45-2.38 (m, 1H, H_{b}C(2)). ¹³C NMR: (125 MHz, CDCl₃) 147.7 (C(2′)), 139.2 (C(1′)), 133.9 (C(3)), 133.4 (C(6′)), 128.1 (C(3′)), 128.0 (C(4′)), 124.4 (C(5′)), 119.0 (C(4)), 68.1 (C(1)), 42.8 (C2)). IR: (NaCl) 3417 (m), 3077 (w), 2979 (w), 2915 (w), 1641 (m), 1609 (m), 1578 (m), 1523 (s), 1519 (s), 1444 (m), 1434 (m), 1347 (s), 1298 (m), 1187 (w), 1054 (m), 988 (m), 920 (m), 856 (m), 821 (m). HRMS: (Cl) calcd for C₁₀H₁₂NO₃, 194.0817; found, 194.0815. TLC: R_{f} 0.42 (hexanes/ethyl acetate, 3:1) [UV, CAM]. (Tan, Shen, B.: Deng, W.; Zhao, H.; Liu, L.: Guo, Q.-X. Org. Let. 2003, 5, 1833-1835.)

### Example 19

### Preparation of Methyl 4-(1-hydroxy-3-butenyl)benzoate (1s)

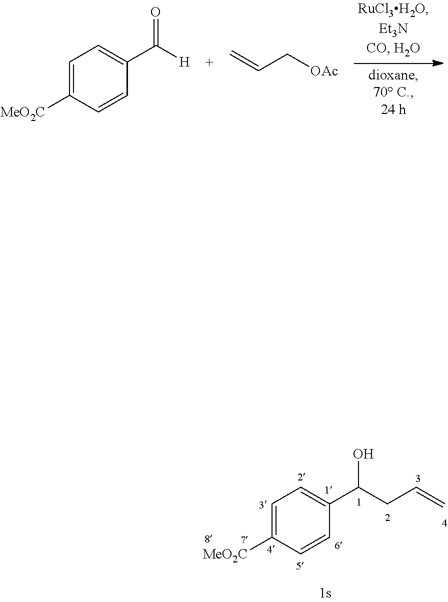

Following the procedure of Example 1, methyl-4-formyl-benzoate (148 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (4:1, 400 mL) to provide 1s (165 mg, 87%) as a colorless oil.

¹H NMR: (500 MHz, CDCl₃) 7.99 (d, J=8.0, 2H, Aryl), 7.40 (d, J=8.0, 2H, Aryl), 5.77 (m, 1H, HC(3)), 5.16-5.12 (m, 2H, H₂C(4)), 4.78 (dd, J=7.5, 4.5, 1 H, HC(1)), 3.87 (s, 3H, H₃C(8′)), 2.53-2.40 (m, 3H, H₂C(2) & HO). ¹³C NMR: (125 MHz, CDCl₃) 166.9 (C(7′)), 149.0 (C(1′)), 133.8 (C(3)), 129.6 (C(3′) & C(5′)), 129.2 (C(4′)), 125.7 (C(2′) & C(6′)), 118.8 (C(4)), 72.7 (C(1)), 52.0 (C(8′)), 43.7 (C2)). IR: (NaCl) 3440 (m), 3077 (w), 2951 (m), 2910 (m), 1722 (s), 1641 (m), 1611 (m), 1576 (w), 1436 (s), 1415 (m), 1281 (s), 1192 (m), 1113 (s). HRMS: (ESI) calcd for C₁₂H₁₅O₃, 207.1021; found, 207.1019. TLC: R_{f} 0.35 (hexanes/ethyl acetate, 3:1) [UV, CAM]. (Tan, X.-H.; Shen, B.; Deng, W.; Zhao, H.; Liu, L.; Guo, Q.-X. Org. Let, 2003, S, 1833-1835.; Furstner, A.; Voigtlander, D. Synthesis, 2000, 7, 959-969.)

### Example 20

### Preparation of (E)-1-Phenyl-1,5-hexadien-3-ol (1t)

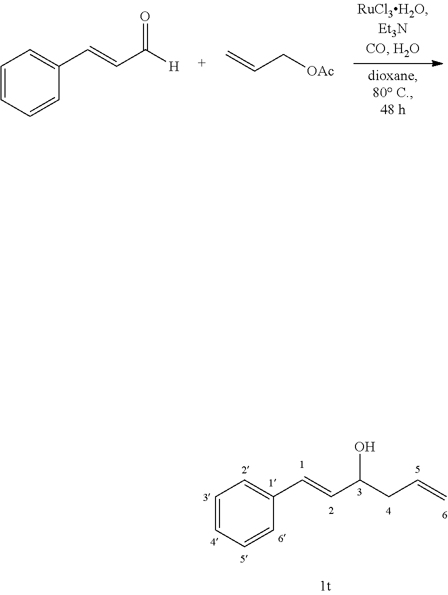

Following the procedure of Example 1, cinnamaldehyde (132 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (4:1, 100 mL; 3:1, 200 mL) to provide 1t (137 mg, 79%) as a colorless oil.

¹H NMR: (400 MHz, CDCl₃) 7.42-7.39 (m, 2H, Aryl), 7.35-7.32 (m, 2H, Aryl), 7.28-7.25 (m, 1H, Aryl), 6.63 (d, J=15.8, 1H, HC(1)), 6.27 (dd, J=15.8, 6.4, 1 H, HC(2)), 5.80 (dddd, J=18.1, 10.2, 7.5, 6.9, 1H, HC(5)), 5.23-5.18 (m, 2H, H₂C(6)), 4.39 (tdd, J=6.0, 6.4, 4.0, 1H, HC(3)), 2.48-2.37 (m, 2H, H₂C(4)), 1.93 (d, J=4.0, 1H, OH). ¹³C NMR: (125 MHz, CDCl₃) 136.6 (C(1′)), 134.0 (C(5)), 131.5 (C(2)), 130.3 (C(1)), 128.5 (C(3′) & C(5′)), 127.6 (C(4′)), 126.4 (C(3′) & C((5′)), 118.4 (C(6)), 71.6 (C(3)), 41.9 (C(4)) IR: (NaCl) 3367 (m), 3077 (m), 3025 (m), 2904 (m), 1640 (m), 1599 (w), 1493 (m), 1448 (m), 1126 (m), 1029 (m), 966 (s). HRMS: (Cl) calcd for C₁₂H₁₅₀, 175.1123; found, 175.1122. TLC: R_{f} 0.48 (hexanes/ethyl acetate, 3:1) [UV, CAM]. (Ishiyama, T.; Ahiko, T.; Miyaura, N. J. Am. Chem. Soc. 2002, 124, 12414-12415.)

### Example 21

### Preparation of (E)-2-Methyl-1-phenyl-1,5-hexadien-3-ol (1u)

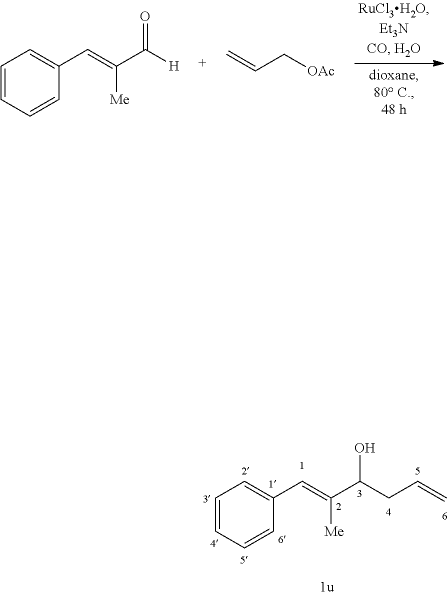

Following the procedure of Example 1, α-methyl-E-cinnamaldehyde (146 mg, 1 mmol), RuCl₃.xH₂O (6.2 mg, 0.03 mmol, 0.03 equiv), allyl acetate (163 μL, 1.5 mmol, 1.5 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 35 psi of CO at 80° C. for 48 h. Silica gel column chromatography was eluted with hexanes/MTBE (5:1, 400 mL) to provide 1u (148 mg, 79′)/0) as a pale yellow oil.

¹H NMR: (500 MHz, CDCl₃) 7.38-7.20 (m, 5H, Aryl), 6.52 (br, 1H, HC(1)), 5.83 (m, 1H, HC(5)), 5.20 (d, J=15.3, 1H, HₐC(6)), 5.17 (d, J=8.5, 1H, H_{b}C(6)), 4.24 (quint, J=5.5, 1H, HC(3)), 2.52-2.40 (m, 2H, H₂C(4)), 1.91 (s, 3H, H₃C(7′)), 1.87 (d, J=5.5, 1H, HO). ¹³C NMR: (125 MHz, CDCl₃) 139.5 (C(2′)), 137.5 (C(1′)), 134.5 (C(5)), 128.9 (C(3′) & C(5′)), 128.1 (C(2′) & C(6′)), 126.4 (C(4′)), 125.7 (C(1)), 118.0 (C(6)), 76.5 (C(3)), 40.1 (C(4)), 13.6 (C(7′)). IR: (NaCl) 3390 (m), 3071 (m), 3018 (m), 2977 (m), 2914 (m), 1640 (m), 1599 (m), 1490 (m), 1442 (m), 1325 (m), 1041 (s), 997 (s), 916 (s). HRMS: (Cl) calcd for C₁₃H₁₇O, 189.1279; found, 189.1280. TLC: R_{f} 0.40 (hexanes/MTBE, 4:1) [UV, CAM]. (Chretien, J-M.; Zammattio, F.; Gauthier, D.; Grognec, E. L.; Paris, M.; Quintard, J-P. Chem. Eur. J. 2006, 12, 6816-6828.)

### Example 22

### Preparation of 1-Cyclohexenyl-3-buten-1-ol (1v)

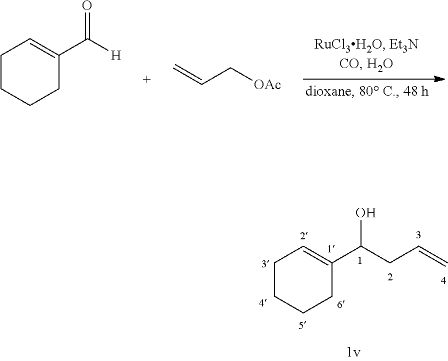

Following the procedure of Example 1, 1-cyclohexene-1-carboxaldehyde (110 mg, 1 mmol), RuCl₃.xH₂O (6.2 mg, 0.03 mmol, 0.03 equiv), allyl acetate (163 μL, 1.5 mmol, 1.5 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 35 psi of CO at 80° C. for 48 h. Silica gel column chromatography was eluted with pentane/MTBE (10:1, 100 mL; 4:1, 200 mL); the solvent was removed by rotary evaporation at 5-10° C., 25 torr, to provide 1v (118 mg, 78′)/0) as a colorless oil.

¹H NMR: (500 MHz, CDCl₃) 5.78 (ddt, J=17.0, 10.2, 7.4, 1H, HC(3)), 5.67 (br, 1H, HC2′)), 5.14-5.08 (m, 2H, H₂C(4)), 4.00 (m, 1H, HC(1)), 2.36-2.25 (m, 2H, H₂C(2)), 2.10-1.88 (m, 4H, H₂C(3′) & H₂C(6′)), 1.68-1.51 (m, 5H, H₂C(4′) & H₂C(5′) & HO). ¹³C NMR: (125 MHz, CDCl₃) 139.2 (C(1′)), 134.9 (C(3), 123.0 (C(2′)), 117.6 (C(4)), 75.2 (C(1)), 39.8 (C(2)), 24.9 (C(3′)), 23.8 (C(6′)), 22.6 (C5′)), 22.5 (C(4′)). IR: (NaCl) 3366 (m), 3075 (m), 2928 (s), 2853 (s), 1641 (m), 1436 (m), 1297 (m), 1137 (m), 1030 (m), 912 (s), 842 (m). HRMS: (Cl) calcd for C₁₀H₁₅O, 151.1123; found, 151.1120. TLC: R_{f} 0.48 (hexanes/MTBE, 4:1) [I₂, CAM]. (Kimura, M.; Shimizu, M.; Tanaka, S.; Tamaru, Y. Tetrahedron 2005, 61, 3709-3718.)

### Example 23

### Preparation of Nonen-4-ol (1w)

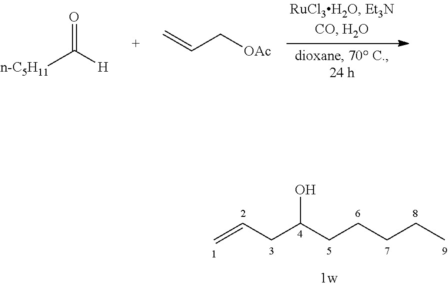

Following the procedure of Example 1, hexanal (100 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. The reaction mixture was transferred to a separatory funnel, diluted with 10 mL of diethyl ether (10 mL), washed with water (5 mL). The aqueous layer was extracted with diethyl ether (10 mL×2). The combined organic fractions were combined, dried over MgSO₄. The solvent was removed by rotary evaporation at 5-10° C., 25 mm Hg. Silica gel column chromatography was eluted with pentane/MTBE (4:1, 200 mL) to provide 1w (122 mg, 86%) as a colorless oil.

¹H NMR: (400 MHz, CDCl₃) 5.82 (m, 1H, HC(2)) 5.15-5.11 (m, 2H, H₂C(1)), 3.64 (m, 1H, HC(4)), 2.33-2.09 (m, 2H, H₂C(3)), 1.62 (br, 1H, HO), 1.48-1.25 (m, 8H, H₂C(5-8), 0.88 (t, J=6.8, 3H, H₃C(9)). ¹³C NMR: (125 MHz, CDCl₃) 134.9 (C(2)), 118.0 (C(1)), 70.7 ((C(4)), 41.9 (C(3)), 36.7 (C(5)), 31.8 (C(7)), 25.3 (C6)), 22.6 (C8)), 14.0 (C9)). IR: (NaCl) 3366 (m), 3076 (m), 2929 (s), 2859 (s), 1640 (m), 1467 (m), 1119 (m), 1024 (m), 995 (m), 912 (m). HRMS: (Cl) calcd for C₉H₁₉O, 143.1436; found, 143.1434. TLC: R_{f} 0.44 (hexanes/MTBE, 4:1) [12]. (Kimura, M.; Shimizu, M.; Tanaka, S.; Tamaru, Y. Tetrahedron 2005, 61, 3709-3718.)

### Example 24

### Preparation of 1-Phenyl-5-hexen-3-ol (1x)

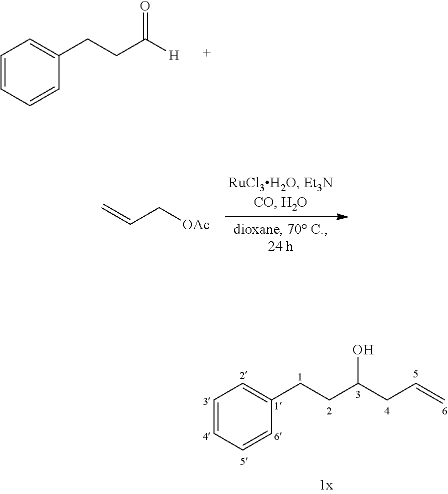

Following the procedure of Example 1, hydrocinnamaldehyde (134 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with pentane/MTBE (4:1, 300 mL) to provide 1×(158 mg, 90%) as a colorless oil.

¹H NMR: (400 MHz CDCl₃) 7.32-7.18 (m, 5H, Aryl), 5.83 (dddd, J=16.0, 10.0, 7.6, 6.0, 1H, HC(5)), 5.18-5.13 (m, 2H, H₂C(6)), 3.71-3.65 (m, 1H, HC(3)), 2.83 (dt, J=13.6, 7.6, 1H, HₐC(1)), 2.66 (dt, J=13.6, 8.4, 1H, H_{b}C(1)), 2.33 (dtt, J=14.0, 5.2, 1.2, 1H, HₐC(4)), 2.20 (dtt, J=14.0, 8.0, 0.8, 1H, H_{b}C(4)), 1.82-1.72 (m, 2H, H₂C(2)), 1.73 (br, 1H, HO). ¹³C NMR: (125 MHz, CDCl₃) 142.0 (C(1′)), 134 (C(5)), 128.4 (C(3′) & C(5′)), 128.3 (C(2′) & C(4′)), 125.8 (C(4′)), 118.3 (C(6)), 69.8 (C(3)), 42.0 (C(4)), 38.4 (C(2)), 32.0 (C(1)). IR: (NaCl) 3390 (s), 3062 (m), 3026 (s), 2929 (s), 2860 (m), 1640 (m), 1602 (m), 1494 (s), 1454 (s), 1047 (s), 916 (s), 864 (m). HRMS: (Cl) calcd for C₁₂H₁₇O, 177.1280; found, 177.1282. TLC: R_{f} 0.35 (hexanes/MTBE, 4:1) [UV, CAM]. (Furstner, A.; Voigtlander, D. Synthesis, 2000, 7, 959-969.; Kimura, M.; Shimizu, M.; Tanaka, S.; Tamaru, Y. Tetrahedron 2005, 61, 3709-3718.)

### Example 25

### Preparation of 1-Cyclohexyl-3-buten-1-ol (1y)

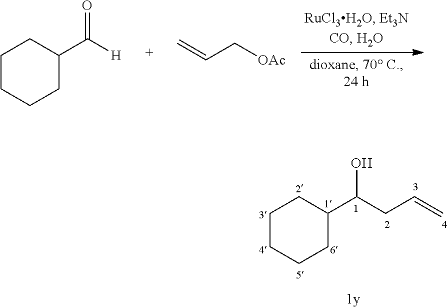

Following the procedure of Example 1, cyclohexanecarboxaldehyde (112 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with hexanes/MTBE (4:1, 200 mL) to provide 1y (126 mg, 82%) as a colorless oil.

¹H NMR: (500 MHz, CDCl₃) 5.89-5.79 (m, 1H, HC(3)), 5.14 (d, J=15.6, 1H, HₐC(4)), 5.13 (d, J=10.5, 1H, H_{b}C(4)), 3.40 (m, 1H, HC(1)), 2.29-2.38 (dt, J=14.6, 7.6, 1H, HₐC(2)), 2.13 (dt, J=14.6, 8.2, 1H, H_{b}C(2)), 1.87-1.60 (m, 6H, c-hexyl & OH), 1.39-0.99 (m, 6H, c-hexyl). ¹³C NMR: (125 MHz CDCl₃) 135.4 (C(3)), 117.8 (C(4)), 74.7 (C(1)), 43.0 (C(2)), 38.8 (C(1′)), 29.0 (c-hexyl), 28.1 (c-hexyl), 26.5 (c-hexyl), 26.2 (c-hexyl), 26.1 (c-hexyl). IR: (NaCl) 3390 (m), 3075 (w), 2925 (s), 2852 (s), 1640 (m), 1449 (m), 1036 (m), 985 (m), 910 (m). HRMS: (Cl) calcd for C₁₀H₁₉O, 155.1436; found, 155.1438. TLC: R_{f} 0.39 (hexanes/MTBE, 4:1) [12]. (Tan, X.-H.; Shen, B.; Deng, W.; Zhao, H.; Liu, L.; Guo, Q.-X. Org. Let. 2003, 5, 1833-1835.; Furstner, A.; Voigtlander, D. Synthesis, 2000, 7, 959-969.; Shen, K.-H.; Yao, C.-F. J. Org. Chem. 2006, 71, 3980-3983.)

### Example 26

### Preparation of 2,2-Dimethyl-5-hexen-3-ol (1z) [SN9-47]

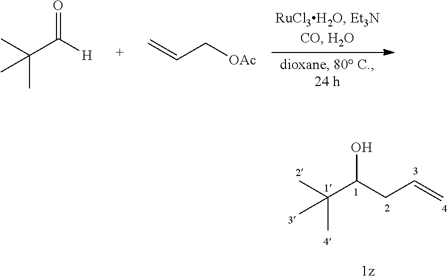

Following the procedure of Example 1, pivaldehyde (86 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 80° C. for 24 h. The reaction mixture was transferred to a separatory funnel, diluted with 10 mL of diethyl ether (10 mL), washed with water (5 mL). The aqueous layer was extracted with diethyl ether (10 mL×2). The combined organic fractions were combined, dried over MgSO₄. The solvent was removed by distillation at 760 mm Hg. The yellow residue was purified by silica gel column chromatography, eluted with pentane/diethyl ether (10:1, 100 mL; 5:1, 200 mL). The solvent was removed by simple distillation followed by purging the residue with a stream of nitrogen to provide 1z (92 mg, 72%) as a colorless (volatile) oil.

¹H NMR: (500 MHz, CDCl₃) 5.87 (ddd, J=17.0, 10.0, 6.5, 1H, HC(3)), 5.17-5.12 (m, 2H, H₂C(4)), 3.24 (ddd, J=10.7, 3.4, 2.2, 1H, HC(1)), 2.40-2.35 (m, 1H, HₐC(2)), 2.02-1.95 (m, 1H, H_{b}C(2)), 1.59 (d, J=3.4, 1H, HO), 0.96 (s, 9H, t-Butyl). ¹³C NMR: (125 MHz, CDCl₃), 136.6 (C(3)), 117 (C(4)), 78.1 (C(1)), 36.6 (C(2)), 34.6 (C(1′)), 25.7 (C(2′), C(3′), C(4′)). IR: (NaCl) 3346 (m), 3019 (w), 2923 (s), 2853 (s), 1596 (w), 1476 (m), 1455 (m), 1376 (m), 1261 (m), 1023 (w). TLC: R_{f} 0.42 (hexanes/MTBE, 4:1) [12]. (Tan, X.-H.; Shen, B.; Deng, W.; Zhao, H.; Liu, L; Guo, Org. Let. 2003, 5, 1833-1835.; Shen, K.-H.; Yao, C.-F. J. Org. Chem. 2006, 71, 3980-3983.)

### Example 27

### Preparation of 1-(2,2-Dimethyl-1,3-dioxolan-4-yl)-3-buten-1-ol (2)

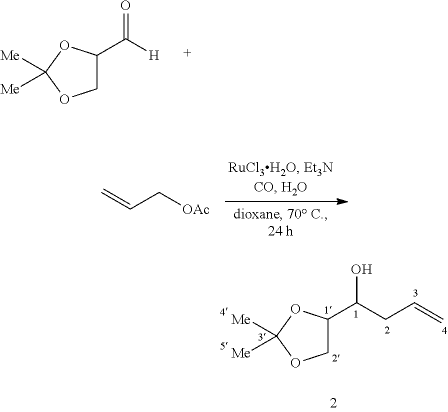

Following the procedure of Example 1, (D)-glyceraldehyde acetonide (98 mg, 1 mmol), RuCl₃.xH₂O (6.2 mg, 0.03 mmol, 0.03 equiv), allyl acetate (130 μL, 1.2 mmol, 1.2 equiv), H₂O (27 μL, 1.5 mmol, 1.5 equiv), Et₃N (14 μL, 0.1 mmol, 0.1 equiv), hexamethylbenzene (16 mg, 0.1 mmol, internal standard for NMR analysis) and dioxane (2.5 mL) were combined under 30 psi of CO at 70° C. for 24 h. Silica gel column chromatography was eluted with pentane/MTBE (3:1, 400 mL) to provide 2 (111 mg, 86%, dr 1.6:1, ratio based on NMR integration) as a colorless oil. (D)-glyceraldehyde acetonide was prepared according to Schmid C. R.; Bryant J. D. Organic Syntheses, Coll. Vol. 9, 450-453.

¹H NMR: (500 MHz, CDCl₃) 5.58-5.56 (m, 1H), 5.04-5.02 (m, 2H), 4.01-3.59 (m, 2H), 3.59-3.58 (m, 0.6H), 3.56-3.55 (m, 1H), 3.52-3.51 (m, 0.4H), 2.73-2.28 (m, 2H), 1.43 (s, 1.2H), 1.42 (s, 1.8H), 1.366 (s, 1.2H), 1.359 (s, 1.8H). ¹³C NMR: (100 MHz, CDCl₃) the erythro isomer (major): 133.9 (C(3)), 118.3 (C(4)), 109.0 (C(3′)), 78.0 (C(1′)), 70.3 (C(1)), 65.1 (C(2′)), 37.5 (C(2)), 26.5 (C(4′)), 25.2 (C(5′)); the threo isomer (minor): 133.9 (C(3)), 117.8 (C(4)), 109.3 (C(3′)), 78.4 (C(1′)), 71.5 (C(1)), 66.0 (C(2′)), 38.2 (C(2)), 26.5 (C(4′)), 25.3 (C(5′)). IR: (NaCl) 3444 (m), 3077 (w), 2986 (s), 2934 (m), 1894 (m), 1642 (m), 1455 (w), 1434 (w), 1381 (m), 1371 (s), 1254 (m), 1214 (s), 1158 (m), 1065 (s). HRMS: (Cl) calcd for C₉H₁₇₀₃, 173.1178; found, 173.1176. TLC: R_{f} 0.17 (hexanes/MTBE, 4:1) [CAM]. ((a) Roush, W. R.; Walts, A. E.; Hoong, L. K. J. Am. Chem. Soc. 1985, 107, 8186-8190; (b) Cossy, J.; Willis, C.; Bellosta, V.; Bouzbouz, S. J. Org. Chem. 2002, 67, 1982-1992.)

### Example 28

### Preparation of 3-Methyl-1-phenylbut-3-en-1-ol (3)

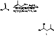

Following the procedure of Example 1, benzaldehyde (102 μL, 106 mg, 1.00 mmol, d=1.045), Ru₃(CO)₁₂ (6.40 mg, 0.01 mmol), tetrabutyl ammonium bromide (8.30 mg, 0.03 mmol), methallyl acetate (215 μL, 186 mg, 1.60 mmol, d=0.865), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 75° C. for 20 h. Silica gel column chromatography was eluted with Et₂O/hexane (15% v/v, 200 mL, 20% v/v, 150 mL) to provide the title compound 3 (107 mg, 66′)/0) as a colorless oil.

¹H NMR: (500 MHz, CDCl₃) 7.41-7.25 (m, 5H, Aryl), 4.93 (m, 1H, CH₂), 4.87 (m, 1H, CH₂), 4.82 (t, J=7.0 Hz, CH(OH)), 2.43 (dd, J=7.0, 0.5 Hz, CH₂), 2.20 (s, 1H, OH), 1.81 (s, 3H). (S. Kobayashi, K. Nishio, J. Org. Chem. 1994, 59(22), 6620-6628.)

### Example 29

### Preparation of 2,2-dimethyl-1-phenylbut-3-en-1-ol (4)

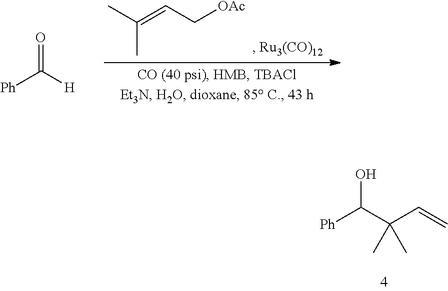

Following the procedure of Example 1, benzaldehyde (102 μL, 106 mg, 1.00 mmol, d=1.045), Ru₃(CO)₁₂ (19.0 mg, 0.03 mmol), tetrabutyl ammonium bromide (25.0 mg, 0.09 mmol), 3-methyl-2-butenylacetate (167 μL, 154 mg, 1.20 mmol, d=0.920), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 85° C. for 43 h. Silica gel column chromatography was eluted with Et₂O/hexane (10% v/v, 400 mL) to provide 4 (52 mg, 29%) as a colorless oil.

¹H NMR: (500 MHz, CDCl₃) 7.36-7.25 (m, 5H, Aryl), 5.93 (dd, J=17.5, 10.5 Hz, 1H, CH), 5.16 (dd, J=10.5, 1.0 Hz, 1H, cis-CH₂), 5.10 (dd, J=17.5, 1.5 Hz, trans-CH₂), 4.44 (d, J=2.5 Hz, CH(OH)), 2.05 (d, J=3.0 Hz, OH), 1.03 (s, 3H), 0.99 (s, 3H). (S. Kobayashi, K. Nishio, J. Org. Chem. 1994, 59(22), 6620-6628.)

### Example 30

### Preparation of (1S,2R)-1,2-diphenylpent-3-en-1-ol (γ-6) and (E)-2-methyl-1,4-diphenylbut-3-en-1-ol (α-6)

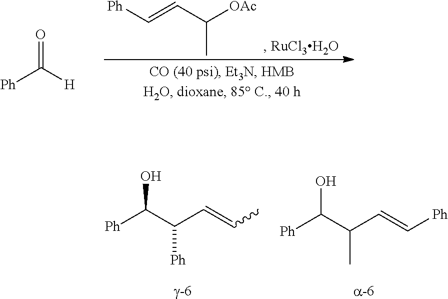

Following the procedure of Example 1, benzaldehyde (51.0 μL, 53.0 mg, 1.00 mmol, d=1.045), RuCl₃.xH₂O (3.90 mg, 0.01 mmol), 3-acetoxy-2-phenyl-1-butene (114 mg, 0.60 mmol), H₂O (14 μL, 0.75 mmol, d=1.000), Et₃N (7.0 μL, 0.05 mmol, d=0.726), hexamethylbenzene (8.0 mg, 0.05 mmol) and dioxane (1.2 mL) were combined under 40 psi of CO at 85° C. for 43 h. Silica gel column chromatography was eluted with Et₂O/hexane (10% v/v, 400 mL, 20% v/v, 150 mL) to provide γ-6 (20 mg, 17%, anti:syn=1:1.7) and α-6 (45 mg, 38%, anti:syn=1:1.6) as a colorless oils.

Data for α-6: ¹H NMR: (500 MHz, CDCl₃) 7.41-7.09 (m, 10H, syn and anti Aryl), 6.55 (d, J=16 Hz, 1H, anti-CH), 6.40 (d, J=16 Hz, syn-CH), 6.20 (dd, J=16, 8.5 Hz, anti-CH), 6.13 (dd, J=16, 7.5 Hz, syn-CH), 4.71 (d, J=5.0 Hz, syn-CH(OH)), 4.46 (d, J=8.0 Hz, anti-CH(OH)), 2.78-2.62 (m, syn and anti-CH(CH₃)), 2.19 (s, 1H, OH), 2.02 (s, 1H, OH), 1.12 (d, J=5.5 Hz, 3H, syn-CH₃), 0.97 (d, J=5.5 Hz, 3H, anti-CH₃).

Data for γ-6: ¹H NMR: (500 MHz, CDCl₃) 7.41-7.09 (m, 10H, syn and anti Aryl), 5.97-5.84 (m, 2H, syn and anti CH), 5.82-5.68 (m, 2H, syn and anti CH), 4.84 (d, J=7.5 Hz, 1H, anti-CH(OH)), 4.77 (d, J=8.0 Hz, 1H, syn-CH(OH)), 3.91 (dd, J=8.5, 8.5 Hz, 1H, anti-CH(CH₃)), 3.48 (dd, J=8.5, 8.5 Hz, 1H, syn-CH(CH₃)), 2.43 (d, J=1.5 Hz, 1H, syn-OH), 2.32 (d, J=2.0 Hz, 1H, anti-OH), 1.76 (dd, J=6.5, 1.5 Hz, 3H, syn-CH₃), 1.62 (dd, J=7.0, 1.5 Hz, 3H, anti-CH₃). (T. Hayashi, Y. Matsumoto, T. Kiyoi, Y. Ito, S. Kohra, Y. Tominaga, A. Hosomi, Tetrahedron Lett., 1988, 29(44), 5667-5670.)

### Example 31

### Preparation of 1-phenyl-2-vinylpropane-1,3-diol (α-8) and 5-phenylpent-2-ene-1,5-diol (γ-8)

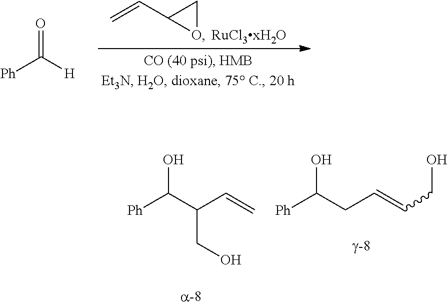

Following the procedure of Example 1, benzaldehyde (102 μL, 106 mg, 1.00 mmol, d=1.045), RuCl₃.xH₂O (7.80 mg, 0.03 mmol), vinyl oxirane (97.0 μL, 84.0 mg, 1.20 mmol, d=0.87), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 75° C. for 20 h. Silica gel column chromatography was eluted with EtOAc/hexane (50% v/v, 300 mL, 80% v/v, 100 mL) to provide α-8 (23 mg, 12%, anti:syn 3.0:1) and γ-8 (88 mg, 49%, E:Z 16:1) as a colorless oils.

Data for α-8: ¹H NMR: (500 MHz, CDCl₃) 7.38-7.25 (m, 10H, anti and syn Aryl), 5.81 (ddd, J=17, 10, 9.0 Hz, 1H, anti-CH), 5.60 (ddd, J=17, 11, 8.5 Hz, 1H, syn-CH), 5.27 (dd, J=10, 1.5 Hz, 1H, anti-CH₂), 5.17 (dd, J=17, 1.5 Hz, 1H, anti-CH₂), 5.06 (dd, J=10, 1.5 Hz, 1H syn-CH₂), 5.02 (dd, J=17, 1.5 Hz, 1H, syn-CH₂), 4.83 (d, J=5.5 Hz, 1H, anti-CH(OH)), 4.79 (d, J=8.0 Hz, 1H, syn-CH(OH)), 3.89-3.76 (m, 2H, syn-CH₂), 3.68-3.58 (m, 2H, anti-CH₂), 2.70-2.55 (m, 2H, anti and syn-CH(CH₂OH)), 1.85 (s, 1H, syn-OH), 1.65 (s, 1H, anti-OH).

Data for γ-8: ¹H NMR: (500 MHz, CDCl₃) 7.38-7.25 (m, 10H, E and Z Aryl), 5.85 (dt, J=11, 7.5 Hz, 1H, Z—CH), 5.61 (dt, J=11, 8.3 Hz, 1H, Z—CH), 5.80-5.65 (m, 2H, E-CH), 4.72 (dd, J=7.0, 5.5 Hz, 1H, E-CH(OH)), 4.73 (dd, J=7.5, 5.0 Hz, 1H, Z—CH(OH)), 4.13 (dd, J=12, 7.5 Hz, 1H, Z—CH₂), 4.02 (dd, J=12, 7.5 Hz, 1H, Z—CH₂), 4.08 (d, J=5.5 Hz, 2H, E-CH₂), 2.65-2.42 (m, 2H, Z—CH₂), 2.49 (t, J=6.5 Hz, 2H, E-CH₂), 2.45 (bs, 1H, E-OH), 2.20 (bs, 1H, Z—OH), 1.95 (bs, 1H, E-OH), 1.80 (bs, 1H, Z—OH). (0. Fujimura, K. Takai, K. Utimoto, J. Org. Chem. 1990, 55(6), 1705-1706.; S. Araki, K. Kameda, J. Tanaka, T. Hirashita, H. Yamamura, M. Kawai, J. Org. Chem. 2001, 66(23), 7919-7921.)

Three variations on this preparation were performed, adjusting the amount of vinyl oxirane allyl donor, the reaction temperature, and the reaction time. In the first variation, benzaldehyde (102 μL, 106 mg, 1.00 mmol, d=1.045), RuCl₃.xH₂O (7.80 mg, 0.03 mmol), vinyl oxirane (194 μL, 168 mg, 2.40 mmol, d=0.87), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 85° C. for 40 h. Silica gel column chromatography was eluted with EtOAc/hexane (50% v/v, 300 mL, 80% v/v, 100 mL) to provide α-8 (12 mg, 7%, anti:syn 2.6:1) and γ-8 (160 mg, 90%, E:Z 10:1) as a colorless oils.

In the second variation, benzaldehyde (102 μL, 106 mg, 1.00 mmol, d=1.045), Ru₃(CO)₁₂ (6.40 mg, 0.01 mmol), tetrabutyl ammonium bromide (8.30 mg, 0.03 mmol), vinyl oxirane (97.0 μL, 84.0 mg, 1.20 mmol, d=0.87), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 75° C. for 20 h. Silica gel column chromatography was eluted with EtOAc/hexane (50% v/v, 300 mL, 80% v/v, 100 mL) to provide γ-8 (160 mg, 90%, E:Z 22:1) as a colorless oil.

In the third variation, benzaldehyde (102 μL, 106 mg, 1.00 mmol, d=1.045), Ru₃(CO)₁₂ (6.40 mg, 0.01 mmol), tetrabutyl ammonium bromide (8.30 mg, 0.03 mmol), vinyl oxirane (161 μL, 140 mg, 2.00 mmol, d=0.87), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 75° C. for 20 h. Silica gel column chromatography was eluted with EtOAc/hexane (50% v/v, 300 mL, 80% v/v, 100 mL) to provide γ-8 (172 mg, 97%, E:Z 23:1) as a colorless oil.

### Example 32

### Preparation of 1,2-diphenylbut-3-en-1-ol (γ-anti-9) and (E)-1,4-diphenylbut-3-en-1-ol (α-E-9)

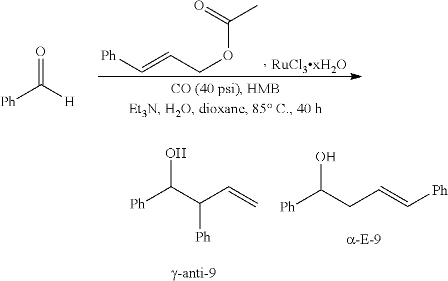

Following the General Procedure, benzaldehyde (102 μL, 106 mg, 1.00 mmol, d=1.045), RuCl₃.xH₂O (7.80 mg, 0.03 mmol), cinnamyl acetate (200 μL, 211 mg, 1.20 mmol, d=1.057), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 85° C. for 40 h. Silica gel column chromatography was eluted with Et₂O/hexane (10% v/v, 200 mL, 20% v/v, 200 mL) to provide γ-anti-9 (103 mg, 47%) and α-E-9 (70 mg, 31%) as a colorless oils.

Data for γ-anti-9: ¹H NMR: (500 MHz, CDCl₃) 7.27-7.04 (m, 10H, Aryl), 6.28 (ddd, J=17, 9.0, 1.5 Hz, 1H, CH), 5.29 (d, J=10 Hz, 1H, cis-CH₂), 5.24 (d, J=17 Hz, 1H, trans-CH₂), 4.86 (d, J=7.5 Hz, 1H, CH(OH)), 3.58 (t, J=8.0 Hz, 1H, CH(Ph)).

Data for α-E-9: ¹H NMR: (500 MHz, CDCl₃) 7.42-7.10 (m, 10H, Aryl), 6.51 (d, J=16 Hz, 1H, CH), 6.25-6.17 (m, 1H, CH), 4.82 (t, J=7.5 Hz, 1H, CH(OH)), 2.71-2.64 (m, 2H, CH₂), 2.15 (d, J=3.5 Hz, OH). (T.-S. Jang, G. Keum, S. B. Kang, B. Y. Chung, Y. Kim, Synthesis, 2003, 5, 775-779.; S. Sebelius, K. J. Szabo, Eur. J. Org. Chem. 2005, 2539-2547.)

In a variation on this preparation, ethanol was used as the solvent. Benzaldehyde (51.0 μL, 53.0 mg, 0.50 mmol, d=1.045), RuCl₃.xH₂O (3.90 mg, 0.01 mmol), cinamyl acetate (100 μL, 105 mg, 0.60 mmol, d=1.057), H₂O (14 μL, 0.75 mmol, d=1.000), Et₃N (7.0 μL, 0.05 mmol, d=0.726), hexamethylbenzene (8.0 mg, 0.05 mmol) and ethanol (1.2 mL) were combined under 40 psi of CO at 85° C. for 40 h. Silica gel column chromatography was eluted with Et₂O/hexane (15% v/v, 400 mL) to provide γ-anti-9 (108 mg, 96%) and α-E-9 (2 mg, 1%) as a colorless oils.

### Example 33

### Preparation of 2-methyl-1-phenylbut-3-en-1-ol (γ-10)

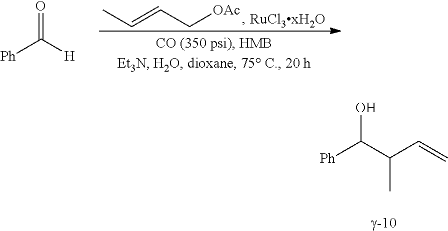

Following the procedure of Example 1, benzaldehyde (102 μL, 106 mg, 1.00 mmol, d=1.045), RuCl₃.xH₂O (7.80 mg, 0.03 mmol), crotyl acetate (150 μL, 137 mg, 1.20 mmol, d=0.919), H₂O (27 μL, 1.50 mmol, d=1.000), Et₃N (14 μL, 0.10 mmol, d=0.726), hexamethylbenzene (16.0 mg, 0.10 mmol) and dioxane (2.5 mL) were combined under 40 psi of CO at 75° C. for 20 h. Silica gel column chromatography was eluted with Et₂O/hexane (10% v/v, 200 mL, 20% v/v, 200 mL) to provide γ-10 (68 mg, 42%, anti:syn 1.6:1) as a colorless oil.

¹H NMR: (500 MHz, CDCl₃) 7.38-7.24 (m, 5H, syn and anti Aryl), 5.86-5.72 (m, 2H, syn and anti CH), 5.24-5.02 (m, 4H, syn and anti CH₂), 4.61 (dd, J=5.0, 4.0 Hz, 1H, syn-CH(OH)), 4.36 (dd, J=8.0, 2.5 Hz, 1H, anti-CH(OH)), 2.61-2.44 (m, 2H, syn and anti CH(CH₃)), 2.18 (d, J=2.5 Hz, 1H, anti-OH), 1.98 (d, J=3.5 Hz, 1H, syn-OH), 1.01 (d, J=7.0 Hz, 3H, syn-CH₃), 0.87 (d, J=6.5 Hz, 3H, anti-CH₃). (W. R. Roush, K. Ando, D. B. Powers, A. D. Palkowitz, R. L. Halternan, J. Am. Chem. Soc. 1990, 112(17), 6339-6348.)

Three variations on this preparation were performed using various crotyl carbonyl substances as the allyl donor. In the first variation, benzaldehyde (51.0 μL, 53.0 mg, 0.50 mmol, d=1.045), RuCl₃.xH₂O (3.90 mg, 0.01 mmol), crotyl carbonate (86.4 mg, 0.60 mmol), H₂O (14 μL, 0.75 mmol, d=1.000), Et₃N (7.0 μL, 0.05 mmol, d=0.726), hexamethylbenzene (8.0 mg, 0.05 mmol) and dioxane (1.2 mL) were combined under 40 psi of CO at 75° C. for 20 h. Silica gel column chromatography was eluted with Et₂O/hexane (15% v/v, 400 mL) to provide γ-10 (64 mg, 79%, anti:syn 1:1.1) as a colorless oil.

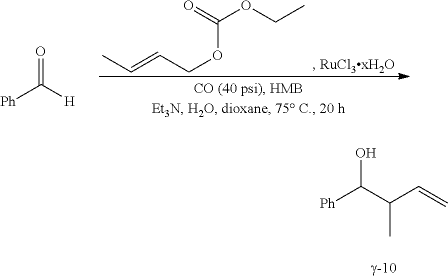

In a second variation, crotyl benzoate was added as the first reaction component. Benzaldehyde (51.0 μL, 53.0 mg, 0.50 mmol, d=1.045), Ru₃(C0)₁₂ (3.20 mg, 0.01 mmol), TBACl (4.10 mg, 0.01 mmol) crotyl benzoate (106 mg, 0.60 mmol), H₂O (14 μL, 0.75 mmol, d=1.000), Et₃N (7.0 μL, 0.05 mmol, d=0.726), hexamethylbenzene (8.0 mg, 0.05 mmol) and dioxane (1.2 mL) were combined under 40 psi of CO at 75° C. for 20 h. Silica gel column chromatography was eluted with Et₂O/hexane (15% v/v, 400 mL) to provide γ-10 (63 mg, 78%, anti:syn 1.8:1) as a colorless oil.

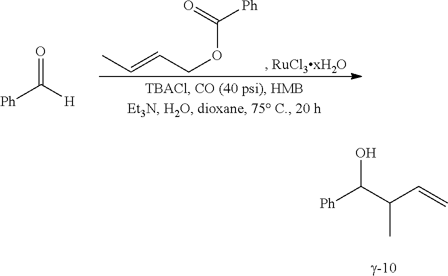

In a third variation, benzaldehyde (51.0 μL, 53.0 mg, 0.50 mmol, d=1.045), RuCl₃.xH₂O (3.90 mg, 0.01 mmol), crotyl acetate (75.0 μL, 68.0 mg, 0.60 mmol, d=0.919), H₂O (14 μL, 0.75 mmol, d=1.000), Et₃N (7.0 μL, 0.05 mmol, d=0.726), hexamethylbenzene (8.0 mg, 0.05 mmol) and ethanol (1.2 mL) were combined under 40 psi of CO at 75° C. for 20 h. Silica gel column chromatography was eluted with Et₂O/hexane (15% v/v, 400 mL) to provide γ-10 (67 mg, 83%, anti:syn 1:2.8) as a colorless oil.

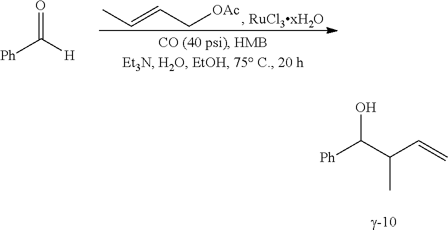

In a fourth variation, benzaldehyde (102 μL, 106 mg, 1.00 mmol, d=1.045), RuCl₃.xH₂O (7.80 mg, 0.03 mmol), 1-methylallyl acetate (153 μL, 137 mg, 1.20 mmol, d=0.894), H₂O (27 pt, 1.50 mmol, d=1.000), Et₃N (14 pt, 0.10 mmol, d=0.726), hexamethylbenzene (16.0 mg, 0.10 mmol) and dioxane (2.5 mL) were combined under 40 psi of CO at 75° C. for 20 h. Silica gel column chromatography was eluted with Et₂O/hexane (15% v/v, 200 mL, 20% v/v, 200 mL) to provide γ-10 (121 mg, 75%, anti:syn 1.9:1) as a colorless oil.

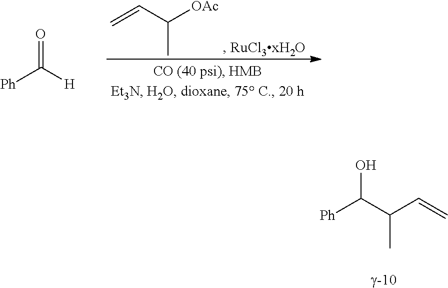

### Example 34

### Larger Scale Preparation of 5-phenylpent-2-ene-1,5-diol (γ-8)

Preparation of 5-phenylpent-2-ene-1,5-diol (γ-8) was done in 10 mmol scale. The data obtained were completely consistent with those of the 1 mmol scale reaction of Example 31.

At ambient temperature open to the air, 80 mL glass cylindrical glass vessel was charged with triruthenium dodecacarbonyl (64.0 mg, 0.10 mmol), internal standard hexamethyl benzene (162 mg, 1.00 mmol), tetrabutylammonium chloride (83.0 mg, 0.30 mmol) and dioxane (25.0 mL). The clear orange colored heterogenous mixture was further treated with water (0.27 mL, 270 mg, 1.50 mmol, d=1.00), triethylamine (0.14 mL, 101 mg, 1.00 mmol, d=0.726), benzaldehyde (1.01 mL, 1.06 g, 10.0 mmol, d=1.04) and vinyl oxirane (1.61 mL, 1.40 g, 20.0 mmol, d=0.87). The glass vessel was sealed with a glass lid which was secured using a teflon tape, placed in metal sleeve and charged with CO (100 psi) 5 times, each time pressure was released. Finally, pressure of CO was adjusted to 40 psi and the vessel was inserted into a Rocker Assembly. The parameters were set as follows: ramp temperature: 1 h; temperature of the reaction: 75° C., reaction time: 20 h. After the 20 h period the system was allowed to cool to rt over 4 h. The gas was vented to the atmospheric pressure. The pale yellow, clear reaction mixture was transferred to a round bottom flask, the vessel rinsed with CHCl₃. The combined solution was concentrated in vacuo. The residue was purified by chromatography on a silica gel column (eluting with EtOAc/hexane 50% v/v, 800 mL, 80% v/v, 1000 mL) to provide γ-8 (1.726 g, 97%; E:Z ratio 10:1) as a colorless oil. ¹H NMR spectrum of this product matched completely with that of the product from Example 31.

### Example 35

### Larger Scale Preparation of 1-(2-Methoxyphenyl)-3-buten-1-ol (1d)

Preparation of 1-(2-Methoxyphenyl)-3-buten-1-ol (1d) was done in 10 mmol scale. The data obtained were completely consistent with those of the 1 mmol scale reaction of Example 4.

Following the procedures of Example 34, using the same apparatus, by use of ruthenium trichloride (78.5 mg, 0.30 mmol), internal standard hexamethyl benzene (162 mg, 1.00 mmol), dioxane (25.0 mL), water (0.27 mL, 270 mg, 1.50 mmol, d=1.00), triethylamine (0.14 mL, 101 mg, 1.00 mmol, d=0.726), allyl acetate (1.30 mL, 1.20 g, 12.0 mmol, d=0.928) and 2-methoxybenzaldehyde (1.36 g, 10.0 mmol), after purification by chromatography on a silica gel column (eluting with EtOAc/hexane 10% v/v, 1000 mL, 20% v/v, 1000 mL), the title compound 1d (1.723 g, 97%) as a colorless oil was obtained. ¹H NMR spectrum of this product matched completely with that of the product from the 1 mmol scale reaction of Example 4.

While various embodiments of the invention have been described, it will be apparent to those of ordinary skill in the art that other embodiments and implementations are possible within the scope of the invention. Accordingly, the invention is not to be restricted except in light of the attached claims and their equivalents.

1. A method of performing a chemical reaction, comprising:
reacting an allyl donor and a substrate selected from the group consisting of an aldehyde and a hemiacetal in a reaction mixture,
the reaction mixture comprising
a ruthenium catalyst, a halide, carbon monoxide at a level of at least 1 equivalent relative to the substrate, water at a level of at least 1 equivalent relative to the substrate, and an amine at a level of from 0.01 to 0.5 equivalent relative to the substrate; or
a halide-free ruthenium catalyst, carbon monoxide at a level of at least 1 equivalent relative to the substrate, water at a level of at least 1 equivalent relative to the substrate, and an amine at a level of from 0 to 0.5 equivalent relative to the substrate, where the reaction mixture does not include a halide;
where the reacting comprises maintaining the reaction mixture at a temperature of at least 40° C.; and
forming a homoallylic alcohol in the reaction mixture.

2. The method of claim 1, where the reaction mixture comprises a ruthenium catalyst, a halide, carbon monoxide at a level of at least 1 equivalent relative to the substrate, water at a level of at least 1 equivalent relative to the substrate, and an amine at a level of from 0.01 to 0.5 equivalent relative to the substrate, and
where the ruthenium catalyst comprises the halide.

3. The method of claim 2, where the ruthenium catalyst is selected from the group consisting of RuCl₃, [Cp*RuCl₂]ₙ, [(COD)RuCl₂]ₙ, [Ru(CO)₃C12]2, and allylRu(CO)₃Br.

4. The method of claim 1, where the reaction mixture comprises a ruthenium catalyst, a halide, carbon monoxide at a level of at least 1 equivalent relative to the substrate, water at a level of at least 1 equivalent relative to the substrate, and an amine at a level of from 0.01 to 0.5 equivalent relative to the substrate, and
where the ruthenium catalyst is a halide-free ruthenium catalyst, and the halide is present in the reaction mixture as a halide salt.

5. The method of claim 1, where the reaction mixture comprises a ruthenium catalyst, a halide, carbon monoxide at a level of at least 1 equivalent relative to the substrate, water at a level of at least 1 equivalent relative to the substrate, and an amine at a level of from 0.01 to 0.5 equivalent relative to the substrate, and
where the number of equivalents of amine is within 30% of the number of equivalents of the halide.

6. The method of claim 1, where the reaction mixture comprises a halide-free ruthenium catalyst, carbon monoxide at a level of at least 1 equivalent relative to the substrate, water at a level of at least 1 equivalent relative to the substrate, and an amine at a level of from 0 to 0.5 equivalent relative to the substrate, where the reaction mixture does not include a halide.

7. The method of claim 6, where the halide-free ruthenium catalyst is selected from the group consisting of Ru₃(CO)₁₂, allylRu(CO)₃OAc, allylRu(CO)₃OBz, and (Et₄N)₂[Ru₆C(CO)₁₆].

8. The method of claim 1, where the amine is present at a level of from 0.1 to 0.5 equivalent relative to the substrate.

9. The method of claim 1, where the ruthenium catalyst is present at a level providing from 0.01 to 0.03 equivalent of Ru relative to the substrate.

10. The method of claim 1, where the substrate is an aldehyde selected from the group consisting of benzaldehyde, 4-methoxybenzaldehyde, 3-methoxybenzaldehyde, 2-methoxybenzaldehyde, 4-dimethylaminobenzaldehyde, 2-hydroxybenzaldehyde, 2-bromobenzaldehyde, 4-methylbenzaldehyde, 2-methylbenzaldehyde, 2,4,6-trimethylbenzaldehyde, 1-naphthylaldehyde, 2-furaldehyde, 2-thiophenecarboxaldehyde, N-tosyl-pyrrole-2-carboxaldehyde, 4-(trifluoromethyl)benzaldehyde, 4-nitrobenzaldehyde, 3-nitrobenzaldehyde, 2-nitrobenzaldehyde, methyl-4-formyl-benzoate, cinnamaldehyde, α-methyl-E-cinnamaldehyde, 1-cyclohexene-1-carboxaldehyde, hexanal, hydrocinnamaldehyde, cyclohexanecarboxaldehyde, pivaldehyde, and (D)-glyceraldehyde acetonide.

11. The method of claim 1, where the substrate is a hemiacetal selected from the group consisting of tetrahydro-2H-pyran-2-ol and tetrahydrofuran-2-ol.

12. The method of claim 1, where the allyl donor is present at a level of 1.0 to 1.5 equivalents relative to the substrate.

13. The method of claim 1, where the allyl donor is selected from the group consisting of allyl acetate, vinyl oxirane, allyl alcohol, diallyl carbonate, allyl formate, a α,γ-disubstituted allyl acetate, a γ,γ-disubstituted allyl acetate, a β-substituted allyl acetate, a cinnamyl ester, a crotyl ester, and 1-methylallyl acetate.

14. The method of claim 1, where the carbon monoxide is present at a level of from 1 to 5 equivalents relative to the substrate.

15. The method of claim 1, where the carbon monoxide is present at a pressure of from 15 to 200 psi.

16. The method of claim 1, where the water is present at a level of from 1 to 2 equivalents relative to the substrate.

17. The method of claim 1, where the reaction mixture further comprises a solvent.

18. The method of claim 1, where the yield of the homoallylic alcohol in the reaction mixture after maintaining the reaction mixture at a temperature of at least 40° C. for at least 8 hours is from 70% to 100%.

19. The method of claim 1, where the reacting comprises maintaining the reaction mixture at a temperature of from 70° C. to 100° C.

20. The method of claim 19, where the yield of the homoallylic alcohol in the reaction mixture after maintaining the reaction mixture at a temperature of from 70° C. to 100° C. for at least 8 hours is from 70% to 100%.
